(19) United States
(12) Patent Application Publication
(10) Pub. No.: US 2018/0055369 A1
(43) Pub. Date: Mar. 1, 2018

(54) LAYERED SENSING INCLUDING RF-ACOUSTIC IMAGING

(71) Applicant: QUALCOMM Incorporated, San Diego, CA (US)

(72) Inventors: David William Burns, San Jose, CA (US); Jonathan Charles Griffiths, Fremont, CA (US); Yipeng Lu, Davis, CA (US)

(21) Appl. No.: 15/253,407

(22) Filed: Aug. 31, 2016

(51) Int. Cl. A61B 5/00; G01S 15/89; A61B 5/024; A61B 5/145; A61B 10/00

(52) U.S. Cl. CPC A61B 5/0095; G01S 15/8965; A61B 5/6898; A61B 5/7425; A61B 2576/00; A61B 5/14532; A61B 5/14542; A61B 10/0041; A61B 5/02438

(57) ABSTRACT

An apparatus or a system may include an ultrasonic sensor array, a radio frequency (RF) source system and a control system. Some implementations may include a light source system and/or an ultrasonic transmitter system. The control system may be capable of controlling the RF source system to emit RF radiation and of receiving signals from the ultrasonic sensor array corresponding to acoustic waves emitted from portions of a target object in response to being illuminated with the RF radiation. The control system may be capable of acquiring ultrasonic image data from the acoustic wave emissions received from the target object.

### TECHNICAL FIELD

This disclosure relates generally to biometric imaging devices and methods, including but not limited to biometric devices and methods applicable to mobile devices.

### DESCRIPTION OF THE RELATED TECHNOLOGY

Medical diagnostic and monitoring devices are generally expensive, difficult to use and invasive. Imaging blood vessels, blood and other sub-epidermal tissues can be particularly challenging. For example, using ultrasonic technology to image such features can be challenging due to the small acoustic impedance contrast between many types of bodily tissues. In another example, imaging and analysis of oxygenated hemoglobin with direct ultrasonic methods can be very difficult because of the low acoustic contrast between oxygenated and oxygen-depleted blood.

### SUMMARY

The systems, methods and devices of the disclosure each have several innovative aspects, no single one of which is solely responsible for the desirable attributes disclosed herein.

One innovative aspect of the subject matter described in this disclosure can be implemented in an apparatus. The apparatus may include an ultrasonic sensor array, a radio frequency (RF) source system and a control system. In some implementations, a mobile device may be, or may include, the apparatus. For example, a mobile device may include a biometric system as disclosed herein.

The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system may be capable of controlling the RF source system to emit RF radiation. In some instances, the RF radiation may induce first acoustic wave emissions inside a target object. In some examples, the control system may be capable of acquiring first ultrasonic image data from the first acoustic wave emissions received by the ultrasonic sensor array from the target object. According to some examples, the control system may be capable of selecting a first acquisition time delay for the reception of acoustic wave emissions primarily from a first depth inside the target object.

In some examples, the apparatus may include a platen. According to some such examples, the platen may be coupled to the ultrasonic sensor array. In some instances, the target object may be positioned on, or proximate, a surface of the platen.

In some implementations, the RF source system may include an antenna array capable of emitting RF radiation at one or more frequencies in the range of about 10 MHz to about 60 GHz. In some examples, “approximately” or “about” as used herein may mean within +/−5%, whereas in other examples “approximately” or “about” may mean within +/−10%, +/−15% or +/−20%. In some examples, the RF source system may include a broad-area antenna array capable of irradiating the target object with either substantially uniform RF radiation or with focused RF radiation at a target depth. In some implementations, the RF source system may include one or more loop antennas, one or more dipole antennas, one or more microstrip antennas, one or more slot antennas, one or more patch antennas, one or more lossy waveguide antennas, or one or more millimeter wave antennas, the antennas residing on one or more substrates that may be coupled to the ultrasonic sensor array. According to some implementations, wherein RF radiation emitted from the RF source system may be emitted as one or more pulses. In some implementations, each pulse may have a duration of less than 100 nanoseconds, or a duration of less than about 100 nanoseconds.

According to some implementations, the apparatus may include a light source system. In some implementations, the light source system may be capable of emitting infrared (IR) light, visible light (VIS) and/or ultraviolet (UV) light. In some examples, the control system may be capable of controlling the light source system to emit light. The light may, in some instances, induce second acoustic wave emissions inside the target object. In some examples, the control system may be capable of acquiring second ultrasonic image data from the acoustic wave emissions received by the ultrasonic sensor array from the target object. In some examples, light emitted from the light source system may be emitted as one or more pulses. Each pulse may, for example, have a duration of less than about 100 nanoseconds.

In some implementations, the apparatus may include a substrate. According to some examples, the ultrasonic sensor array may reside in or on the substrate. In some examples, at least a portion of the light source system may be coupled to the substrate. According to some implementations, IR light, VIS light and/or UV light from the light source system may be transmitted through the substrate. In some examples, RF radiation emitted by the RF source system may be transmitted through the substrate. In some implementations, RF radiation emitted by the RF source system may be transmitted through the ultrasonic sensor array.

According to some implementations, the apparatus may include a display. In some such implementations, at least some subpixels of the display may be coupled to the substrate. According to some such implementations, the control system may be further capable of controlling the display to depict a two-dimensional image that corresponds with the first ultrasonic image data or the second ultrasonic image data. In some examples, the control system may be capable of controlling the display to depict an image that superimposes a first image that corresponds with the first ultrasonic image data and a second image that corresponds with the second ultrasonic image data. According to some implementations, at least some subpixels of the display may be adapted to detect infrared light, visible light, UV light, ultrasonic waves, and/or acoustic wave emissions.

In some implementations, the control system may be capable of selecting first through N^{th} acquisition time delays and of acquiring first through N^{th} ultrasonic image data during first through N^{th} acquisition time windows after the first through N^{th} acquisition time delays. Each of the first through N^{th} acquisition time delays may, in some instances, correspond to first through N^{th} depths inside the target object. The control system may be capable of controlling a display to depict a three-dimensional image that corresponds with at least a subset of the first through N^{th} ultrasonic image data.

In some examples, the first ultrasonic image data may be acquired during a first acquisition time window from a peak detector circuit disposed in each of a plurality of sensor pixels within the ultrasonic sensor array. According to some implementations, the ultrasonic sensor array and a portion of the RF source system may be configured in an ultrasonic button, a display module, and/or a mobile device enclosure.

In some implementations, the apparatus may include an ultrasonic transmitter system. According to some such implementations, the control system may be capable of acquiring second ultrasonic image data from insonification of the target object with ultrasonic waves emitted from the ultrasonic transmitter system. In some examples, ultrasonic waves emitted from the ultrasonic transmitter system may be emitted as one or more pulses. Each pulse may, for example, have a duration of less than 100 nanoseconds, or less than about 100 nanoseconds.

Some implementations of the apparatus may include a light source system and an ultrasonic transmitter system. According to some examples, the control system may be capable of controlling the light source system and the ultrasonic transmitter system. In some examples, the control system may be capable of acquiring second acoustic wave emissions, via the ultrasonic sensor array, from the target object in response to RF radiation emitted from the RF source system, light emitted from the light source system, and/or ultrasonic waves emitted by the ultrasonic transmitter system.

Some innovative aspects of the subject matter described in this disclosure can be implemented in a mobile device. In some examples, the mobile device may include an ultrasonic sensor array, a display, a radio frequency (RF) source system, a light source system and a control system. In some implementations, the control system may be capable of controlling the RF source system to emit RF radiation. The RF radiation may, in some instances, induce first acoustic wave emissions inside a target object. According to some implementations, the control system may be capable of acquiring first ultrasonic image data from the first acoustic wave emissions received by the ultrasonic sensor array from the target object.

In some examples, the control system may be capable of controlling the light source system to emit light that may, in some instances, induce second acoustic wave emissions inside the target object. According to some examples, the control system may be capable of acquiring second ultrasonic image data from the acoustic wave emissions received by the ultrasonic sensor array from the target object. In some implementations, the control system may be capable of controlling the display to present an image corresponding to the first ultrasonic image data, an image corresponding to the second ultrasonic image data, or an image corresponding to the first ultrasonic image data and the second ultrasonic image data.

According to some implementations, the display may be on a first side of the mobile device and the RF source system may emit RF radiation through a second and opposing side of the mobile device. In some examples, the light source system may emit light through the second and opposing side of the mobile device.

According to some examples, the mobile device may include an ultrasonic transmitter system. In some examples, the ultrasonic sensor array may include the ultrasonic transmitter system, whereas in other examples the ultrasonic transmitter system may be separate from the ultrasonic sensor array. In some such examples, the control system may be capable of acquiring third ultrasonic image data from insonification of the target object with ultrasonic waves emitted from the ultrasonic transmitter system. According to some such examples, the control system may be capable of controlling the display to present an image corresponding to the first ultrasonic image data, the second ultrasonic image data and/or the third ultrasonic image data. According to some such implementations, the control system may be capable of controlling the display to depict an image that superimposes at least two images. The at least two images may include a first image that corresponds with the first ultrasonic image data, a second image that corresponds with the second ultrasonic image data and/or a third image that corresponds with the third ultrasonic image data.

In some implementations, the control system may be capable of selecting first through N^{th} acquisition time delays and of acquiring first through N^{th} ultrasonic image data during first through N^{th} acquisition time windows after the first through N^{th} acquisition time delays. Each of the first through N^{th} acquisition time delays may, in some instances, correspond to first through N^{th} depths inside the target object. The control system may be capable of controlling a display to depict a three-dimensional image that corresponds with at least a subset of the first through N^{th} ultrasonic image data. In some examples, the first through N^{th} acquisition time delays may be selected to image a blood vessel, a bone, fat tissue, a melanoma, a breast cancer tumor, a biological component, and/or a biomedical condition.

Additional innovative aspects of the subject matter described in this disclosure can be implemented in an apparatus that includes an ultrasonic sensor array, a radio frequency (RF) source system, a light source system and a control system. In some implementations, the control system may be capable of controlling the RF source system to emit RF radiation. The RF radiation may, in some instances, induce first acoustic wave emissions inside a target object. According to some implementations, the control system may be capable of acquiring first ultrasonic image data from the first acoustic wave emissions received by the ultrasonic sensor array from the target object.

In some examples, the control system may be capable of controlling the light source system to emit light that may, in some instances, induce second acoustic wave emissions inside the target object. According to some examples, the control system may be capable of acquiring second ultrasonic image data from the acoustic wave emissions received by the ultrasonic sensor array from the target object. In some implementations, the control system may be capable of performing an authentication process based on data corresponding to both the first ultrasonic image data and the second ultrasonic image data.

According to some examples, the authentication process may include a liveness detection process. In some examples, the ultrasonic sensor array, the RF source system and the light source system may reside, at least in part, in a button area of a mobile device. According to some implementations, the control system may be capable of performing blood oxygen level monitoring, blood glucose level monitoring and/or heartrate monitoring.

Still other innovative aspects of the subject matter described in this disclosure can be implemented in a method of acquiring ultrasonic image data. In some examples, the method may involve controlling a radio frequency (RF) source system to emit RF radiation. In some instances, the RF radiation may induce first acoustic wave emissions inside a target object. According to some examples, the method may involve acquiring, via an ultrasonic sensor array, first ultrasonic image data from the first acoustic wave emissions received by the ultrasonic sensor array from the target object.

In some examples, the method may involve controlling a light source system to emit light. In some instances, the light may induce second acoustic wave emissions inside the target object. According to some examples, the method may involve acquiring, via the ultrasonic sensor array, second ultrasonic image data from the acoustic wave emissions received by the ultrasonic sensor array from the target object.

In some implementations, the method may involve controlling a display to display an image corresponding to the first ultrasonic image data, an image corresponding to the second ultrasonic image data, or an image corresponding to the first ultrasonic image data and the second ultrasonic image data. In some examples, the method may involve performing an authentication process based on data corresponding to both the first ultrasonic image data and the second ultrasonic image data.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in a non-transitory medium having software stored thereon.

For example, the software may include instructions for controlling one or more devices to perform a method of acquiring ultrasonic image data. In some examples, the method may involve controlling a radio frequency (RF) source system to emit RF radiation. In some instances, the RF radiation may induce first acoustic wave emissions inside a target object. According to some examples, the method may involve acquiring, via an ultrasonic sensor array, first ultrasonic image data from the first acoustic wave emissions received by the ultrasonic sensor array from the target object.

In some examples, the method may involve controlling a light source system to emit light. In some instances, the light may induce second acoustic wave emissions inside the target object. According to some examples, the method may involve acquiring, via the ultrasonic sensor array, second ultrasonic image data from the acoustic wave emissions received by the ultrasonic sensor array from the target object.

In some implementations, the method may involve controlling a display to display an image corresponding to the first ultrasonic image data, an image corresponding to the second ultrasonic image data, or an image corresponding to the first ultrasonic image data and the second ultrasonic image data. In some examples, the method may involve performing an authentication process based on data corresponding to both the first ultrasonic image data and the second ultrasonic image data.

### BRIEF DESCRIPTION OF THE DRAWINGS

Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale. Like reference numbers and designations in the various drawings indicate like elements.

FIG. 1 shows an example of components of blood being differentially heated and subsequently emitting acoustic waves.

FIG. 2 is a block diagram that shows example components of an apparatus according to some disclosed implementations.

FIG. 3 is a flow diagram that shows example blocks of some disclosed methods.

FIG. 4A shows an example of a target object being illuminated by incident RF radiation and/or light, and subsequently emitting acoustic waves.

FIGS. 4B-4E show examples of RF source system components.

FIG. 5 shows an example of a mobile device that includes a biometric system as disclosed herein.

FIG. 6A is a flow diagram that includes blocks of a user authentication process.

FIG. 6B shows an example of an apparatus that includes in-cell multi-functional pixels.

FIG. 7 shows examples of multiple acquisition time delays being selected to receive acoustic waves emitted from different depths.

FIG. 8 is a flow diagram that provides additional examples of biometric system operations.

FIG. 9 shows examples of multiple acquisition time delays being selected to receive ultrasonic waves emitted from different depths, in response to a plurality of pulses.

FIGS. 10A-10C are examples of cross-sectional views of a target object positioned on a platen of an apparatus such as those disclosed herein.

FIG. 10D is a cross-sectional view of the target object illustrated in FIGS. 10A-10C.

FIG. 10E shows a series of simplified two-dimensional images that correspond with ultrasonic image data acquired by the processes shown in FIGS. 10A-10C.

FIG. 10F shows an example of a composite image.

FIG. 11 shows an example of a mobile device capable of performing some methods disclosed herein.

FIG. 12 is a flow diagram that provides an example of a method of obtaining and displaying ultrasonic image data via a mobile device.

FIGS. 13A-13C show examples of mobile devices imaging objects of a person's body.

FIG. 14 shows an example of a sensor pixel array.

FIG. 15A shows an example of an exploded view of an ultrasonic sensor system.

FIG. 15B shows an exploded view of an alternative example of an ultrasonic sensor system.

FIG. 16A shows examples of layers of an apparatus according to one example.

FIG. 16B shows an example of a layered sensor stack that includes the layers shown in FIG. 16A.

FIG. 17A shows examples of layers of an apparatus according to another example.

FIG. 17B shows an example of a layered sensor stack that includes the layers shown in FIG. 17A.

FIG. 18 shows example elements of an apparatus such as those disclosed herein.

### DETAILED DESCRIPTION

The following description is directed to certain implementations for the purposes of describing the innovative aspects of this disclosure. However, a person having ordinary skill in the art will readily recognize that the teachings herein may be applied in a multitude of different ways. The described implementations may be implemented in any device, apparatus, or system that includes a biometric system as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players (such as MP3 players), camcorders, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), electronic photographs, electronic billboards or signs, projectors, architectural structures, microwaves, refrigerators, stereo systems, cassette recorders or players, DVD players, CD players, VCRs, radios, portable memory chips, washers, dryers, washer/dryers, parking meters, packaging (such as in electromechanical systems (EMS) applications including microelectromechanical systems (MEMS) applications, as well as non-EMS applications), aesthetic structures (such as display of images on a piece of jewelry or clothing) and a variety of EMS devices. The teachings herein also may be used in applications such as, but not limited to, electronic switching devices, radio frequency filters, sensors, accelerometers, gyroscopes, motion-sensing devices, magnetometers, inertial components for consumer electronics, parts of consumer electronics products, steering wheels or other automobile parts, varactors, liquid crystal devices, electrophoretic devices, drive schemes, manufacturing processes and electronic test equipment. Thus, the teachings are not intended to be limited to the implementations depicted solely in the Figures, but instead have wide applicability as will be readily apparent to one having ordinary skill in the art.

Various implementations disclosed herein may include a biometric system that is capable of excitation via differential heating and ultrasonic imaging of resultant acoustic wave emission. In some examples, the differential heating may be caused by radio frequency (RF) radiation. Such imaging may be referred to herein as “RF-acoustic imaging.” Alternatively or additionally, the differential heating may be caused by light, such as infrared (IR) light, visible light (VIS) or ultraviolet (UV) light. Such imaging may be referred to herein as “photoacoustic imaging.” Some such implementations may be capable of obtaining images from bones, muscle tissue, blood, blood vessels, and/or other sub-epidermal features. As used herein, the term “sub-epidermal features” may refer to any of the tissue layers that underlie the epidermis, including the dermis, the subcutis, etc., and any blood vessels, lymph vessels, sweat glands, hair follicles, hair papilla, fat lobules, etc., that may be present within such tissue layers. Some implementations may be capable of biometric authentication that is based, at least in part, on image data obtained via RF-acoustic imaging and/or via photoacoustic imaging. In some examples, an authentication process may be based on image data obtained via RF-acoustic imaging and/or via photoacoustic imaging, and also on image data obtained by transmitting ultrasonic waves and detecting corresponding reflected ultrasonic waves.

In some implementations, the incident light wavelength or wavelengths emitted by an RF source system and/or a light source system may be selected to trigger acoustic wave emissions primarily from a particular type of material, such as blood, blood cells, blood vessels, blood vasculature, lymphatic vasculature, other soft tissue, or bones. The acoustic wave emissions may, in some examples, include ultrasonic waves. In some such implementations, the control system may be capable of estimating a blood oxygen level, estimating a blood glucose level, or estimating both a blood oxygen level and a blood glucose level.

Alternatively or additionally, the time interval between the irradiation time and the time during which resulting ultrasonic waves are sampled (which may be referred to herein as the acquisition time delay or the range-gate delay (RGD)) may be selected to receive acoustic wave emissions primarily from a particular depth and/or from a particular type of material. For example, a relatively larger range-gate delay may be selected to receive acoustic wave emissions primarily from bones and a relatively smaller range-gate delay may be selected to receive acoustic wave emissions primarily from shallower sub-epidermal features such as blood vessels, blood, muscle tissue features, etc.

Accordingly, some biometric systems disclosed herein may be capable of acquiring images of sub-epidermal features via RF-acoustic imaging and/or via photoacoustic imaging. In some implementations, a control system may be capable of acquiring first ultrasonic image data from acoustic wave emissions that are received by an ultrasonic sensor array during a first acquisition time window that is initiated at an end time of a first acquisition time delay. According to some examples, the first ultrasonic image data may be acquired during the first acquisition time window from a peak detector circuit disposed in each of a plurality of sensor pixels within the ultrasonic sensor array.

According to some examples, the control system may be capable of controlling a display to depict a two-dimensional (2-D) image that corresponds with the first ultrasonic image data. In some instances, the control system may be capable of acquiring second through N^{th} ultrasonic image data during second through N^{th} acquisition time windows after second through N^{th} acquisition time delays. Each of the second through N^{th} acquisition time delays may correspond to a second through an N^{th} depth inside the target object. According to some examples, the control system may be capable of controlling a display to depict a three-dimensional (3-D) image that corresponds with at least a subset of the first through N^{th} ultrasonic image data.

Particular implementations of the subject matter described in this disclosure can be implemented to realize one or more of the following potential advantages. Imaging sub-epidermal features (such as blood vessels, blood, etc.), melanomas, breast cancer tumors or other tumors, etc., using ultrasonic technology alone can be challenging due to the small acoustic impedance contrast between various types of soft tissue. In some RF-acoustic imaging and/or via photoacoustic imaging implementations, a relatively higher signal-to-noise ratio may be obtained for the resulting acoustic wave emission detection because the excitation is via RF and/or optical stimulation instead of (or in addition to) ultrasonic wave transmission. The higher signal-to-noise ratio can provide relatively more accurate and relatively more detailed imaging of blood vessels and other sub-epidermal features. In addition to the inherent value of obtaining more detailed images (e.g., for improved medical determinations and diagnoses of cancer), the detailed imaging of blood vessels and other sub-epidermal features can provide more reliable user authentication and liveness determinations. Moreover, some RF-acoustic imaging and/or via photoacoustic imaging implementations can detect changes in blood oxygen levels, which can provide enhanced liveness determinations. Some implementations provide a mobile device that includes a biometric system that is capable of some or all of the foregoing functionality. Some such mobile devices may be capable of displaying 2-D and/or 3-D images of melanomas, breast cancer tumors and other sub-epidermal features, bone tissue, biological components, etc. A biological component may include, for example, one or more constituents of blood, body tissue, bone matter, cellular structures, organs, inborn features or foreign bodies.

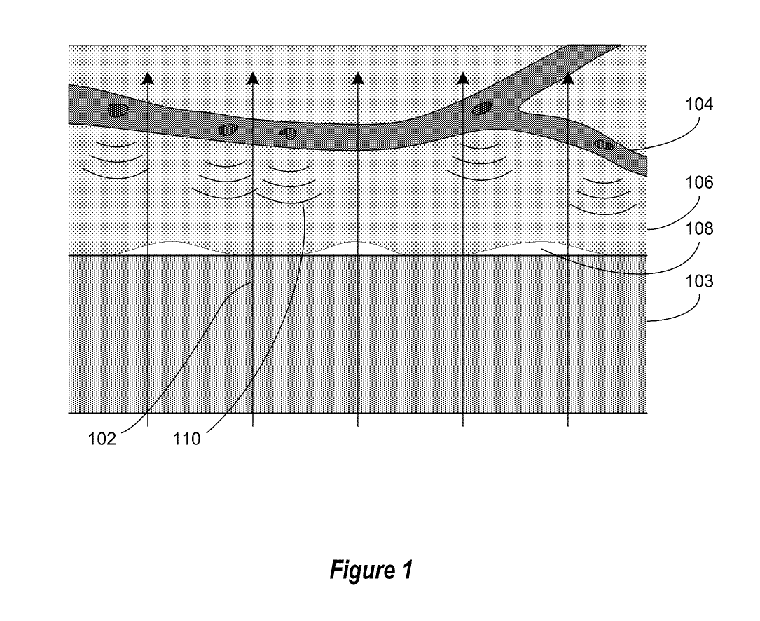

FIG. 1 shows an example of components of blood being differentially heated and subsequently emitting acoustic waves. In this example, incident radiation 102 has been transmitted from a source system (not shown) through a substrate 103 and into a blood vessel 104 of an overlying finger 106. In some examples, the incident radiation 102 may include incident RF radiation from an RF source system. Alternatively or additionally, the incident radiation 102 may include incident light from a light source system. The surface of the finger 106 includes ridges and valleys, so some of the incident radiation 102 has been transmitted through the air 108 in this example. Here, the incident radiation 102 is causing differential excitation of illuminated blood and blood components in the blood vessel 104 (relative to less absorptive blood and blood components in the blood vessel 104) and resultant acoustic wave generation. In this example, the generated acoustic waves 110 include ultrasonic waves.

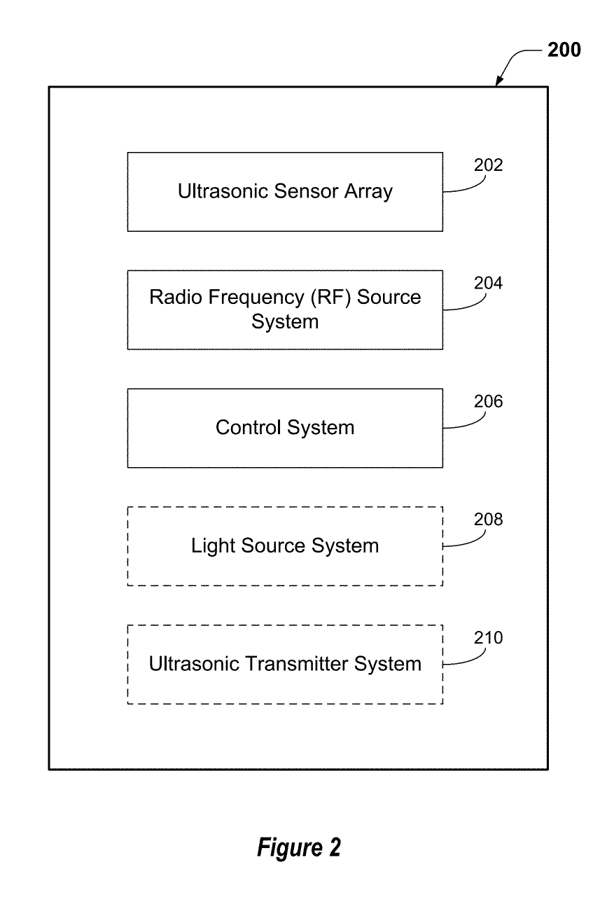

In some implementations, such acoustic wave emissions may be detected by sensors of a sensor array, such as the ultrasonic sensor array 202 that is described below with reference to FIG. 2. In some instances, the incident radiation wavelength, wavelengths and/or wavelength range(s) may be selected to trigger acoustic wave emissions primarily from a particular type of material, such as blood, blood components, blood vessels, other soft tissue, or bones.

FIG. 2 is a block diagram that shows example components of an apparatus according to some disclosed implementations. In this example, the apparatus 200 includes a biometric system. Here, the biometric system includes an ultrasonic sensor array 202, an RF source system 204 and a control system 206. Although not shown in FIG. 2, the apparatus 200 may include a substrate. Some examples are described below. Some implementations of the apparatus 200 may include the optional light source system 208 and/or the optional ultrasonic transmitter system 210. In some examples, the apparatus 200 may include at least one display.

Various examples of ultrasonic sensor arrays 202 are disclosed herein, some of which may include an ultrasonic transmitter and some of which may not. Although shown as separate elements in FIG. 2, in some implementations the ultrasonic sensor array 202 and the ultrasonic transmitter system 210 may be combined in an ultrasonic transceiver. For example, in some implementations, the ultrasonic sensor array 202 may include a piezoelectric receiver layer, such as a layer of PVDF polymer or a layer of PVDF-TrFE copolymer. In some implementations, a separate piezoelectric layer may serve as the ultrasonic transmitter. In some implementations, a single piezoelectric layer may serve as the transmitter and as a receiver. In some implementations, other piezoelectric materials may be used in the piezoelectric layer, such as aluminum nitride (AlN) or lead zirconate titanate (PZT). The ultrasonic sensor array 202 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some alternative examples, the ultrasonic sensor array 202 may be an ultrasonic receiver array and the ultrasonic transmitter system 210 may include one or more separate elements. In some such examples, the ultrasonic transmitter system 210 may include an ultrasonic plane-wave generator, such as those described below.

According to some examples, the RF source system 204 may include an antenna array, such as a broad-area antenna array. The antenna array may, for example, include one or more loop antennas capable of generating low-frequency RF waves (e.g., in the range of approximately 10-100 MHz), one or more dipole antennas capable of generating medium-frequency RF waves (e.g., in the range of approximately 100-5,000 MHz), a lossy waveguide antenna capable of generating RF waves in a wide frequency range (e.g., in the range of approximately 10-60,000 MHz) and/or one or more millimeter-wave antennas capable of generating high-frequency RF waves (e.g., in the range of approximately 3-60 GHz or more). According to some example, the control system 206 may be capable of controlling the RF source system 204 to emit RF radiation in one or more pulses, each pulse having a duration less than 100 nanoseconds, or less than approximately 100 nanoseconds.

In some implementations, the RF source system 204 may include more than one type of antenna and/or a layered set of antenna arrays. For example, the RF source system 204 may include one or more loop antennas. Alternatively or additionally, the RF source system 204 may include one or more dipole antennas, one or more microstrip antennas, one or more slot antennas, one or more patch antennas, one or more lossy waveguide antennas and/or or one or more millimeter wave antennas. According to some such implementations, the antennas may reside on one or more substrates that are coupled to the ultrasonic sensor array.

In some implementations, the control system 206 may be capable of controlling the RF source system 204 to irradiate a target object with substantially uniform RF radiation. Alternatively or additionally, the control system 206 may be capable of controlling the RF source system 204 to irradiate a target object with focused RF radiation at a target depth, e.g., via beamforming.

The control system 206 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 206 may include (and/or be configured for communication with) one or more memory devices, such as one or more random access memory (RAM) devices, read-only memory (ROM) devices, etc. Accordingly, the apparatus 200 may have a memory system that includes one or more memory devices, though the memory system is not shown in FIG. 2.

In this example, the control system 206 is capable of controlling the RF source system 204, e.g., as disclosed herein. The control system 206 may be capable of receiving and processing data from the ultrasonic sensor array 202, e.g., as described below. If the apparatus 200 includes a light source system 208 and/or an ultrasonic transmitter system 210, the control system 206 may be capable of controlling the light source system 208 and/or the ultrasonic transmitter system 210, e.g., as disclosed elsewhere herein. In some implementations, functionality of the control system 206 may be partitioned between one or more controllers or processors, such as a dedicated sensor controller and an applications processor of a mobile device.

Although not shown in FIG. 2, some implementations of the apparatus 200 may include an interface system. In some examples, the interface system may include a wireless interface system. In some implementations, the interface system may include a user interface system, one or more network interfaces, one or more interfaces between the control system 206 and a memory system and/or one or more interfaces between the control system 206 and one or more external device interfaces (e.g., ports or applications processors).

The light source system 208 may, in some examples, include one or more light-emitting diodes. In some implementations, the light source system 208 may include one or more laser diodes. According to some implementations, the light source system may include at least one infrared, optical, red, green, blue, white or ultraviolet light-emitting diode. In some implementations, the light source system 208 may include one or more laser diodes. For example, the light source system 208 may include at least one infrared, optical, red, green, blue or ultraviolet laser diode.

In some implementations, the light source system 208 may be capable of emitting various wavelengths of light, which may be selectable to trigger acoustic wave emissions primarily from a particular type of material. For example, because the hemoglobin in blood absorbs near-infrared light very strongly, in some implementations the light source system 208 may be capable of emitting one or more wavelengths of light in the near-infrared range, in order to trigger acoustic wave emissions from hemoglobin. However, in some examples the control system 206 may control the wavelength(s) of light emitted by the light source system 208 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the ultrasonic sensor array 202. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic sensor array. Image data from the ultrasonic sensor array that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 208 may be capable of emitting a light pulse with a pulse width less than 100 nanoseconds, or less than approximately 100 nanoseconds. In some implementations, the light pulse may have a pulse width between about 10 nanoseconds and about 500 nanoseconds or more. In some implementations, the light source system 208 may be capable of emitting a plurality of light pulses at a pulse frequency between about 1 MHz and about 100 MHz. In some examples, the pulse frequency of the light pulses may correspond to an acoustic resonant frequency of the ultrasonic sensor array and the substrate. For example, a set of four or more light pulses may be emitted from the light source system 208 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 208. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object. In some such implementations, one or more pulses of incident light in the visible range, such as in a red, green or blue wavelength range, may be applied and corresponding ultrasonic images acquired to subtract out background effects.

The apparatus 200 may be used in a variety of different contexts, many examples of which are disclosed herein. For example, in some implementations a mobile device may include the apparatus 200. In some implementations, a wearable device may include the apparatus 200. The wearable device may, for example, be a bracelet, an armband, a wristband, a ring, a headband or a patch. In some examples, a display device may include a display module with multi-functional pixel arrays having ultrasonic, infrared (IR), visible spectrum (VIS), ultraviolet (UV), and/or light-gating subpixels. The ultrasonic subpixels of the display device may detect the photo-acoustic or RF-acoustic wave emissions. Some such examples may provide multiple modalities such as ultrasonic, photo-acoustic, RF-acoustic, optical, IR and UV imaging to provide self-referenced images for biomedical analysis; glucose and blood oxygen levels; detection of skin conditions, tumors, cancerous material and other biomedical conditions; blood analysis; and/or biometric authentication of users. Biomedical conditions may include, for example, a blood condition, an illness, a disease, a fitness level, stress markers, or a wellness level. Various examples are described below.

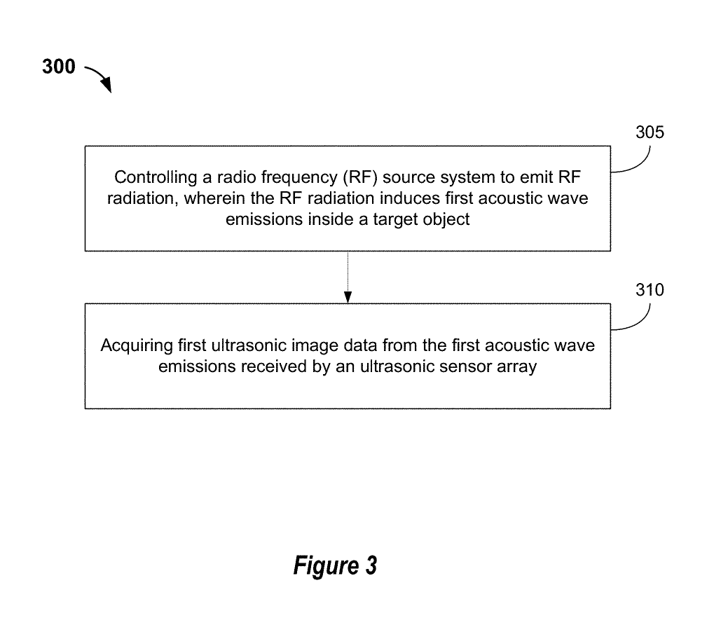

FIG. 3 is a flow diagram that shows example blocks of some disclosed methods. The blocks of FIG. 3 (and those of other flow diagrams provided herein) may, for example, be performed by the apparatus 200 of FIG. 2 or by a similar apparatus. As with other methods disclosed herein, the method outlined in FIG. 3 may include more or fewer blocks than indicated. Moreover, the blocks of methods disclosed herein are not necessarily performed in the order indicated.

Here, block 305 involves controlling an RF source system to emit RF radiation. In some implementations, the control system 206 of the apparatus 200 may control the RF source system 204 to emit RF radiation. According to some examples, the RF source system may include an antenna array capable of emitting RF radiation at one or more frequencies in the range of about 10 MHz to about 60 GHz or more. In some implementations, RF radiation emitted from the RF source system may be emitted as one or more pulses, each pulse having a duration less than 100 nanoseconds, or less than approximately 100 nanoseconds. According to some implementations, the RF source system may include a broad-area antenna array capable of irradiating the target object with substantially uniform RF radiation. Alternatively or additionally, the RF source system may include a broad-area antenna array capable of irradiating the target object with focused RF radiation at a target depth.

In some examples, block 305 may involve controlling an RF source system to emit RF radiation that is transmitted through the ultrasonic sensor array. According to some examples, block 305 may involve controlling an RF source system to emit RF radiation that is transmitted through a substrate and/or other layers of an apparatus such as the apparatus 200.

According to this implementation, block 310 involves receiving signals from an ultrasonic sensor array corresponding to acoustic waves emitted from portions of a target object in response to being illuminated with RF radiation emitted by the RF source system. In some instances the target object may be positioned on a surface of the ultrasonic sensor array or positioned on a surface of a platen that is acoustically coupled to the ultrasonic sensor array. The ultrasonic sensor array may, in some implementations, be the ultrasonic sensor array 202 that is shown in FIG. 2 and described above. One or more coatings or acoustic matching layers may be included with the platen in some examples.

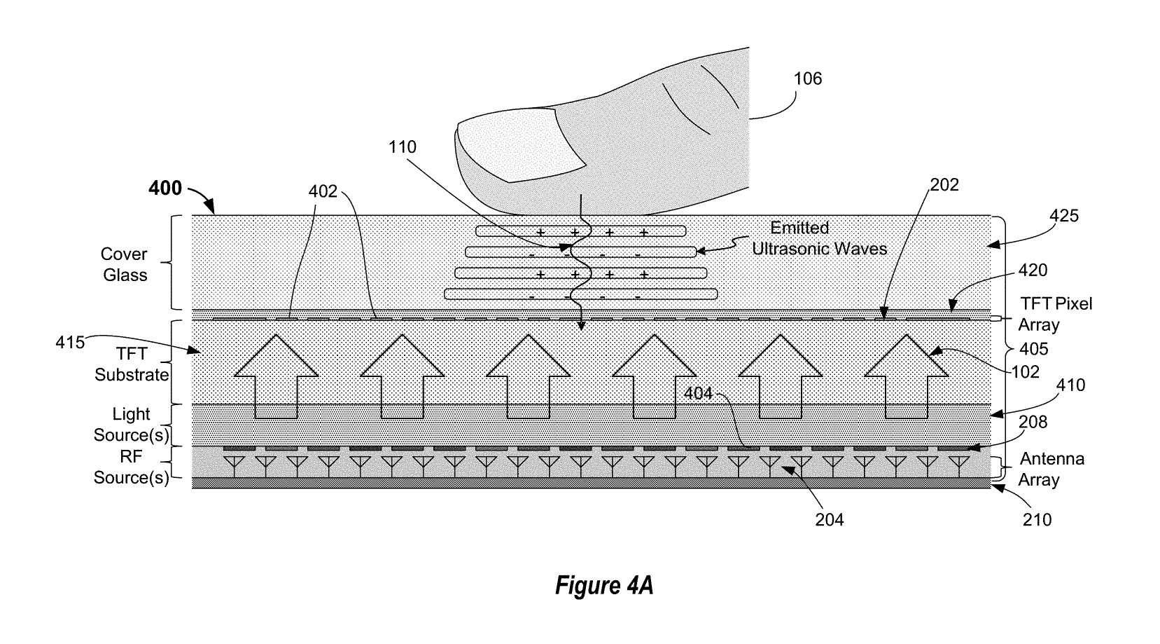

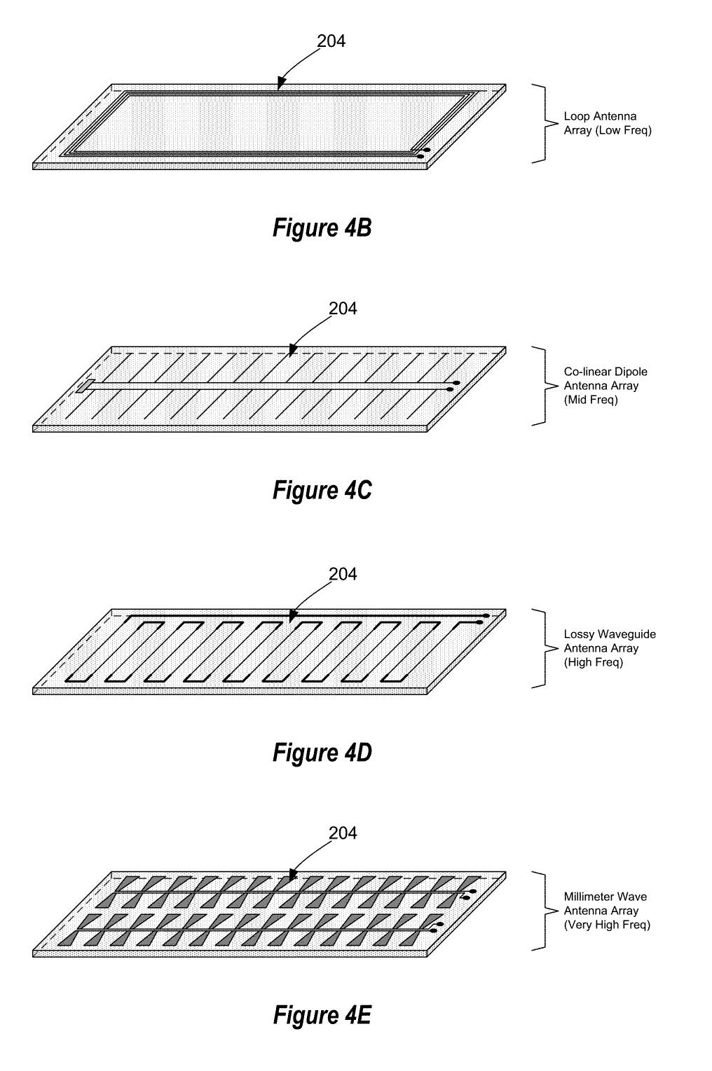

In some examples the target object may be a finger, as shown above in FIG. 1 and as described below with reference to FIG. 4A. However, in other examples the target object may be another body part, such as a palm, a wrist, an arm, a leg, a torso, a head, etc. In some examples the target object may be a finger-like object that is being used in an attempt to spoof the apparatus 200, or another such apparatus, into erroneously authenticating the finger-like object. For example, the finger-like object may include silicone rubber, polyvinyl acetate (white glue), gelatin, glycerin, etc., with a fingerprint pattern formed on an outside surface.

In some examples, the control system may be capable of selecting a first acquisition time delay to receive acoustic wave emissions at a corresponding distance from the ultrasonic sensor array. The corresponding distance may correspond to a depth within the target object. According to some examples, the control system may be capable of receiving an acquisition time delay via a user interface, from a data structure stored in memory, etc.

According to some implementations, the control system may be capable of acquiring first ultrasonic image data from acoustic wave emissions that are received by an ultrasonic sensor array during a first acquisition time window that is initiated at an end time of a first acquisition time delay. According to some examples, the control system may be capable of controlling a display to depict a two-dimensional (2-D) image that corresponds with the first ultrasonic image data. In some instances, the control system may be capable of acquiring second through N^{th} ultrasonic image data during second through N^{th} acquisition time windows after second through N^{th} acquisition time delays. Each of the second through N^{th} acquisition time delays may correspond to second through N^{th} depths inside the target object. According to some examples, the control system may be capable of controlling a display to depict a reconstructed three-dimensional (3-D) image that corresponds with at least a subset of the first through N^{th} ultrasonic image data. Some examples are described below.

As noted above, some implementations may include a light source system. In some examples, the light source system may be capable of emitting infrared (IR) light, visible light (VIS) and/or ultraviolet (UV) light. According to some such implementations, a control system may be capable of controlling the light source system to emit light that induces second acoustic wave emissions inside the target object.

In some examples, the control system may be capable of controlling the light source system to emit light as one or more pulses. Each pulse may, in some examples, have a duration less than 100 nanoseconds, or less than approximately 100 nanoseconds. The control system may be capable of acquiring second ultrasonic image data from the resulting acoustic wave emissions received by the ultrasonic sensor array.

According to some such implementations, the control system may be capable of selecting one or more wavelengths of the light emitted by the light source system. In some implementations, the control system may be capable of selecting a light intensity associated with each selected wavelength. For example, the control system may be capable of selecting the one or more wavelengths of light and light intensities associated with each selected wavelength to generate acoustic wave emissions from one or more portions of the target object. In some examples, the control system may be capable of selecting the one or more wavelengths of light to evaluate one or more characteristics of the target object, e.g., to evaluate blood oxygen levels. Some examples are described elsewhere herein.

As noted above, some implementations of the apparatus 200 include an ultrasonic transmitter system 210. According to some such implementations, the control system 206 may be capable of acquiring ultrasonic image data via insonification of a target object with ultrasonic waves emitted from the ultrasonic transmitter system 210. In some such implementations, the control system 206 may be capable of controlling the ultrasonic transmitter system 210 to emit ultrasonic waves emitted in one or more pulses. According to some such implementations, each pulse may have a duration less than 100 nanoseconds, or less than approximately 100 nanoseconds.

In some examples, the ultrasonic sensor array may reside in or on a substrate. According to some such examples, at least a portion of the light source system may be coupled to the substrate. In some such implementations, method 300 may involve transmitting IR light, VIS light and/or UV light from the light source system through the substrate. According to some implementations, method 300 may involve transmitting RF radiation emitted by the RF source system through the substrate.

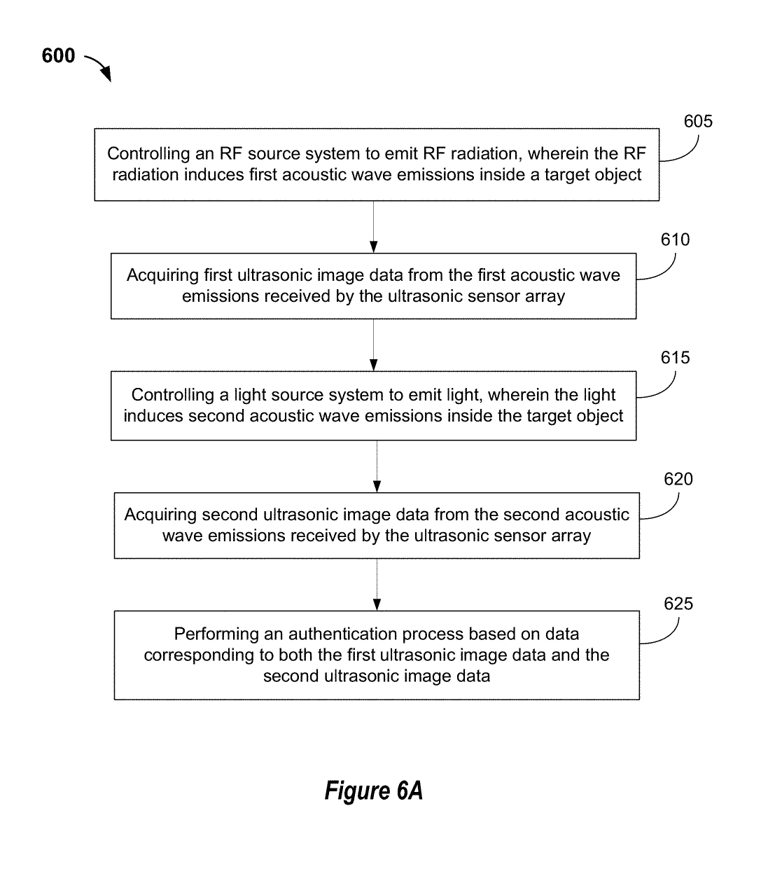

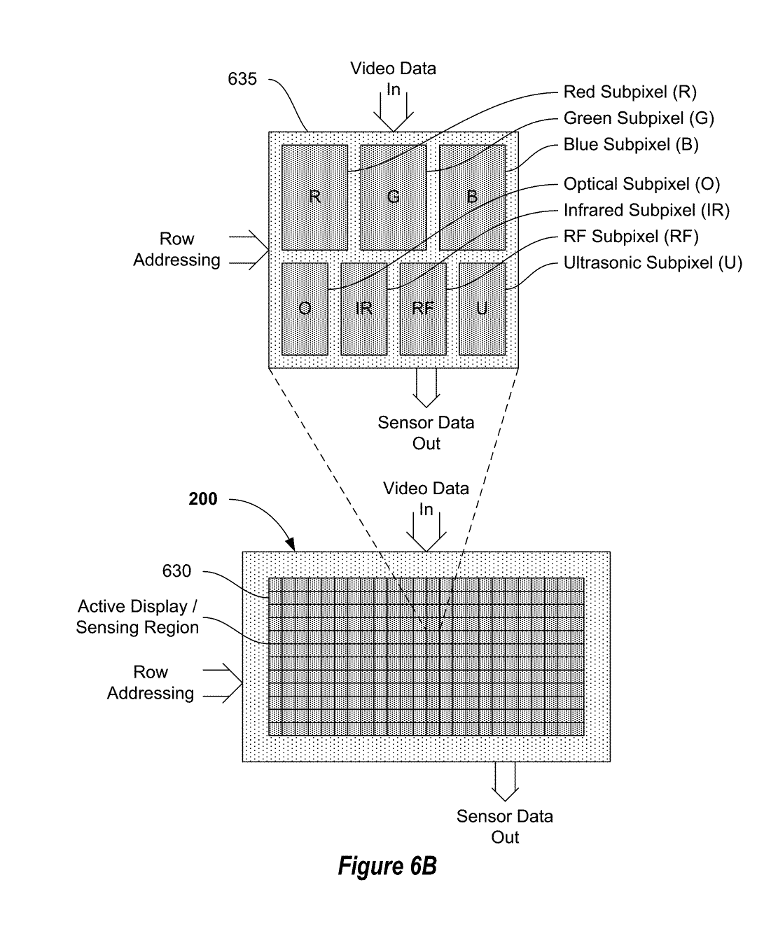

As noted elsewhere herein, some implementations may include at least one display. In some such implementations, the control system may be further capable of controlling the display to depict a two-dimensional image that corresponds with the first ultrasonic image data or the second ultrasonic image data. In some examples, the control system may be capable of controlling the display to depict an image that superimposes a first image that corresponds with the first ultrasonic image data and a second image that corresponds with the second ultrasonic image data. According to some examples, subpixels of the display may be coupled to the substrate. According to some implementations, subpixels of the display may be adapted to detect one or more of infrared light, visible light, UV light, ultrasonic waves, or acoustic wave emissions. Some examples are described below with reference to FIG. 6B.

FIG. 4A shows an example of a cross-sectional view of an apparatus capable of performing the method of FIG. 3. The apparatus 400 is an example of a device that may be included in a biometric system such as those disclosed herein. Although the control system 206 is not shown in FIG. 4A, the apparatus 400 is an implementation of the apparatus 200 that is described above with reference to FIG. 2. As with other implementations shown and described herein, the types of elements, the arrangement of the elements and the dimensions of the elements illustrated in FIG. 4A are merely shown by way of example.

FIG. 4A shows an example of a target object being illuminated by incident RF radiation and/or light, and subsequently emitting acoustic waves. In this implementation, the apparatus 400 includes an RF source system 204, which includes an antenna array in this example. Examples of suitable antenna arrays are described below with reference to FIGS. 4B-4E. In some alternative implementations, the antenna array may include one or more microstrip antennas and/or one or more slot antennas and/or one or more patch antennas. According to some examples, the control system 206 may be capable of controlling the RF source system 204 to emit RF radiation at one or more frequencies in the range of about 10 MHz to about 60 GHz or more. In some examples, the control system 206 may be capable of controlling the RF source system 204 to emit RF radiation in one or more pulses, each pulse having a duration less than about 100 nanoseconds. According to some implementations, the control system 206 may be capable of controlling the RF source system 204 to emit RF radiation that irradiates a target object (such as the finger 106 shown in FIG. 4A) with substantially uniform RF radiation. Alternatively or additionally, the control system 206 may be capable of controlling the RF source system 204 to emit RF radiation that irradiates a target object with focused RF radiation at a target depth.

In this example, the apparatus 400 includes a light source system 208, which may include an array of light-emitting diodes and/or an array of laser diodes. In some implementations, the light source system 208 may be capable of emitting various wavelengths of light, which may be selectable to trigger acoustic wave emissions primarily from a particular type of material. In some instances, the incident light wavelength, wavelengths and/or wavelength range(s) may be selected to trigger acoustic wave emissions primarily from a particular type of material, such as blood, blood vessels, other soft tissue, or bones. To achieve sufficient image contrast, light sources 404 of the light source system 208 may need to have a higher intensity and optical power output than light sources generally used to illuminate displays. In some implementations, light sources with light output of 1-100 millijoules or more per pulse, with pulse widths of 100 nanoseconds or less, may be suitable. In some implementations, light from an electronic flash unit such as that associated with a mobile device may be suitable. In some implementations, the pulse width of the emitted light may be between about 10 nanoseconds and about 500 nanoseconds or more.

In this example, incident radiation 102 has been transmitted from the RF source system 204 and/or the light source system 208 through a sensor stack 405 and into an overlying finger 106. The various layers of the sensor stack 405 may include one or more substrates of glass or other material such as plastic or sapphire that is substantially transparent to the RF radiation emitted by the RF source system 204 and the light emitted by the light source system 208. In this example, the sensor stack 405 includes a substrate 410 to which the RF source system 204 and the light source system 208 are coupled, which may be a backlight of a display according to some implementations. In alternative implementations, the light source system 208 may be coupled to a front light. Accordingly, in some implementations the light source system 208 may be configured for illuminating a display and the target object.

In this implementation, the substrate 410 is coupled to a thin-film transistor (TFT) substrate 415 for the ultrasonic sensor array 202. According to this example, a piezoelectric receiver layer 420 overlies the sensor pixels 402 of the ultrasonic sensor array 202 and a platen 425 overlies the piezoelectric receiver layer 420. Accordingly, in this example the apparatus 400 is capable of transmitting the incident radiation 102 through one or more substrates of the sensor stack 405 that include the ultrasonic sensor array 202 with substrate 415 and the platen 425, which also may be viewed as a substrate. In some implementations, sensor pixels 402 of the ultrasonic sensor array 202 may be transparent, partially transparent or substantially transparent to light and RF radiation, such that the apparatus 400 may be capable of transmitting the incident radiation 102 through elements of the ultrasonic sensor array 202. In some implementations, the ultrasonic sensor array 202 and associated circuitry may be formed on or in a glass, plastic or silicon substrate.

In this example, the portion of the apparatus 400 that is shown in FIG. 4A includes an ultrasonic sensor array 202 that is capable of functioning as an ultrasonic receiver array. According to some implementations, the apparatus 400 may include an ultrasonic transmitter system 210. The ultrasonic transmitter system 210 may or may not be part of the ultrasonic sensor array 202, depending on the particular implementation. In some examples, the ultrasonic sensor array 202 may include PMUT or CMUT elements that are capable of transmitting and receiving ultrasonic waves, and the piezoelectric receiver layer 420 may be replaced with an acoustic coupling layer. In some examples, the ultrasonic sensor array 202 may include an array of pixel input electrodes and sensor pixels formed in part from TFT circuitry, an overlying piezoelectric receiver layer 420 of piezoelectric material such as PVDF or PVDF-TrFE, and an upper electrode layer positioned on the piezoelectric receiver layer sometimes referred to as a receiver bias electrode. In the example shown in FIG. 4A, at least a portion of the apparatus 400 includes an ultrasonic transmitter system 210 that can function as a plane-wave ultrasonic transmitter. The ultrasonic transmitter system 210 may include a piezoelectric transmitter layer with transmitter excitation electrodes disposed on each side of the piezoelectric transmitter layer.

Here, the incident radiation 102 causes excitation within the finger 106 and resultant acoustic wave generation. In this example, the generated acoustic waves 110 include ultrasonic waves. Acoustic emissions generated by the absorption of incident light may be detected by the ultrasonic sensor array 202. A high signal-to-noise ratio may be obtained because the resulting ultrasonic waves are caused by optical stimulation instead of by reflection of transmitted ultrasonic waves.

FIGS. 4B-4E show examples of RF source system components. The RF source system 204 may include one or more of the types of antenna arrays shown in FIGS. 4B-4E. In some examples, the apparatus 200 may include multiple types of antenna arrays, each of which resides on a separate substrate. However, some implementations may include more than one type of antenna array on a single substrate.

In the example shown in FIG. 4B, the RF source system 204 includes a loop antenna array. The loop antenna array may, for example, be capable of generating low-frequency RF waves in the range of approximately 10-100 MHz.

In the example shown in FIG. 4C, the RF source system 204 includes a dipole antenna array. In this implementation, the dipole antenna array is a co-linear dipole antenna array that may, for example, be capable of generating medium-frequency RF waves in the range of approximately 100-5,000 MHz.

In the example shown in FIG. 4D, the RF source system 204 includes a lossy waveguide antenna array. According to some examples, the lossy waveguide antenna array may be capable of generating RF waves in a wide frequency range that includes relatively higher frequencies, e.g., in the range of approximately 10-60,000 MHz.

In the example shown in FIG. 4E, the RF source system 204 includes a millimeter-wave antenna array. Some such antenna arrays are capable of generating RF radiation in a range that includes even higher frequencies, e.g., a range of approximately 3-60 GHz or more.

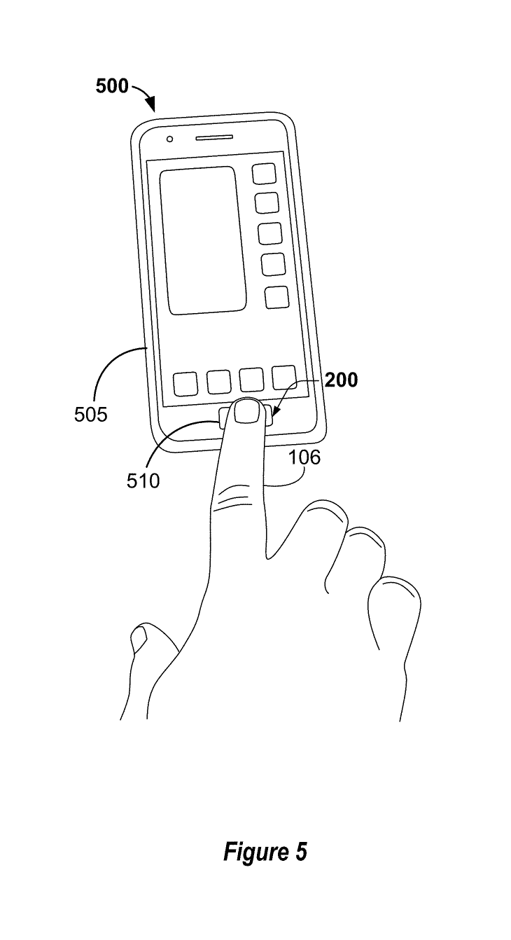

FIG. 5 shows an example of a mobile device that includes a biometric system as disclosed herein. In this example, the mobile device 500 is a smartphone. However, in alternative examples the mobile device 500 may another type of mobile device, such as a mobile health device, a wearable device, a tablet computer, etc.

In this example, the mobile device 500 includes an instance of the apparatus 200 that is described above with reference to FIG. 2. In this example, the apparatus 200 is disposed, at least in part, within the mobile device enclosure 505. According to this example, at least a portion of the apparatus 200 is located in the portion of the mobile device 500 that is shown being touched by the finger 106, which corresponds to the location of button 510. Accordingly, the button 510 may be an ultrasonic button. In some implementations, the button 510 may serve as a home button. In some implementations, the button 510 may serve as an ultrasonic authenticating button, with the ability to turn on or otherwise wake up the mobile device 500 when touched or pressed and/or to authenticate or otherwise validate a user when applications running on the mobile device (such as a wake-up function) warrant such a function.

An RF source system 204 configured for RF-acoustic imaging may reside, at least in part, within the button 510. In some examples, a light source system 208 configured for photoacoustic imaging may reside, at least in part, within the button 510. Alternatively or additionally, an ultrasonic transmitter system 210 configured for insonification of a target object with ultrasonic waves may reside, at least in part, within the button 510.

FIG. 6A is a flow diagram that includes blocks of a user authentication process. In some examples, the apparatus 200 of FIG. 2 may be capable of performing the user authentication process 600. In some implementations, the mobile device 500 of FIG. 5 may be capable of performing the user authentication process 600. As with other methods disclosed herein, the method outlined in FIG. 6A may include more or fewer blocks than indicated. Moreover, the blocks of method 600, as well as other methods disclosed herein, are not necessarily performed in the order indicated.

Here, block 605 involves controlling an RF source system to emit RF radiation. In this example, the RF radiation induces acoustic wave emissions inside a target object in block 605. In some implementations, the control system 206 of the apparatus 200 may control the RF source system 204 to emit RF radiation in block 605. In some examples, the control system 206 may control the RF source system 204 to emit RF radiation at one or more frequencies in the range of about 10 MHz to about 60 GHz or more. According to some such implementations, the control system 206 may be capable of controlling the RF source system 204 to emit at least one RF radiation pulse having a duration of less than 100 nanoseconds, or less than approximately 100 nanoseconds. For example, the control system 206 may be capable of controlling the RF source system 204 to emit at least one RF radiation pulse having a duration of approximately 10 nanoseconds, 20 nanoseconds, 30 nanoseconds, 40 nanoseconds, 50 nanoseconds, 60 nanoseconds, 70 nanoseconds, 80 nanoseconds, 90 nanoseconds, 100 nanoseconds, etc.

In some examples, RF radiation emitted by the RF source system 204 may be transmitted through an ultrasonic sensor array or through one or more substrates of a sensor stack that includes an ultrasonic sensor array. In some examples, RF radiation emitted by the RF source system 204 may be transmitted through a button of a mobile device, such as the button 510 shown in FIG. 5.

In some examples, block 605 (or another block of method 600) may involve selecting a first acquisition time delay to receive the acoustic wave emissions primarily from a first depth inside the target object. In some such examples, the control system may be capable of selecting an acquisition time delay to receive acoustic wave emissions at a corresponding distance from the ultrasonic sensor array. The corresponding distance may correspond to a depth within the target object. According to some such examples, the acquisition time delay may be measured from a time that the RF source system emits RF radiation. In some examples, the acquisition time delay may be in the range of about 10 nanoseconds to about 20,000 nanoseconds or more.

According to some examples, a control system (such as the control system 206) may be capable of selecting the first acquisition time delay. In some examples, the control system may be capable of selecting the acquisition time delay based, at least on part, on user input. For example, the control system may be capable of receiving an indication of target depth or a distance from a platen surface of the biometric system via a user interface. The control system may be capable of determining a corresponding acquisition time delay from a data structure stored in memory, by performing a calculation, etc. Accordingly, in some instances the control system's selection of an acquisition time delay may be according to user input and/or according to one or more acquisition time delays stored in memory.

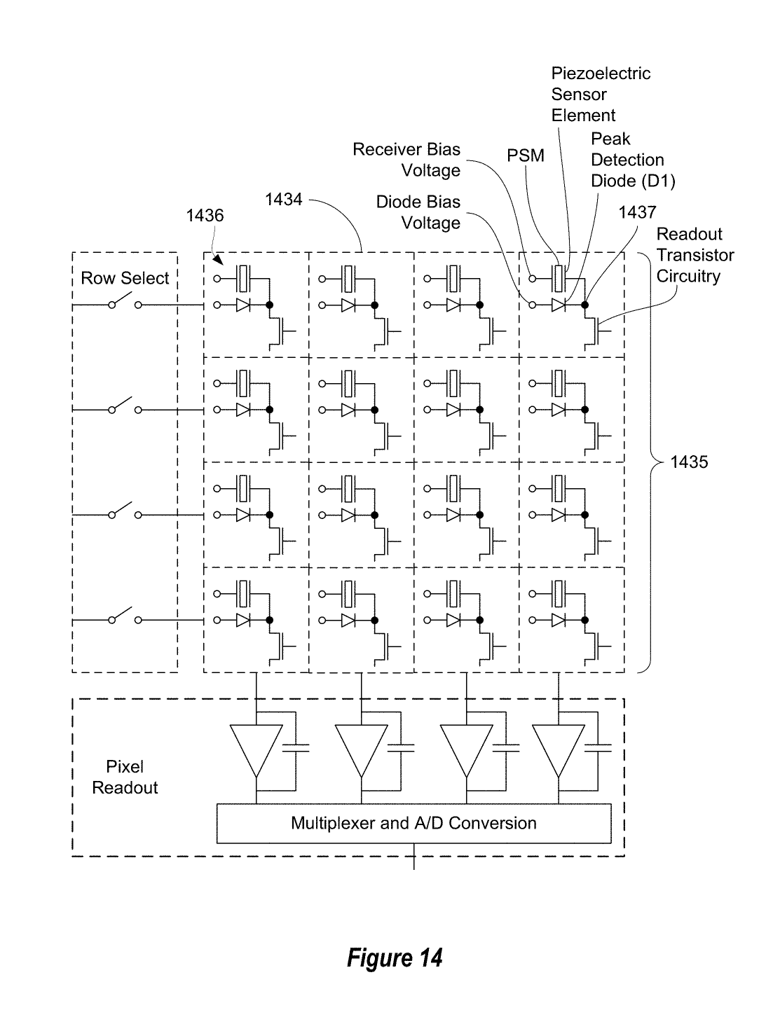

In this implementation, block 610 involves acquiring first ultrasonic image data from the acoustic wave emissions received by an ultrasonic sensor array during a first acquisition time window that is initiated at an end time of the first acquisition time delay. Some implementations may involve controlling a display to depict a two-dimensional image that corresponds with the first ultrasonic image data. According to some implementations, the first ultrasonic image data may be acquired during the first acquisition time window from a peak detector circuit disposed in each of a plurality of sensor pixels within the ultrasonic sensor array. In some implementations, the peak detector circuitry may capture acoustic wave emissions or reflected ultrasonic wave signals during the acquisition time window. Some examples are described below with reference to FIG. 14.

In some examples, the first ultrasonic image data may include image data corresponding to one or more sub-epidermal features, such as vascular image data.

According to this implementation, block 615 involves controlling a light source system to emit light. For example, the control system 206 may control the light source system 208 to emit light. In this example, the light induces second acoustic wave emissions inside the target object. According to some such implementations, the control system 206 may be capable of controlling the light source system 208 to emit at least one light pulse having a duration that is in the range of about 10 nanoseconds to about 500 nanoseconds or more. For example, the control system 206 may be capable of controlling the light source system 208 to emit at least one light pulse having a duration of approximately 10 nanoseconds, 20 nanoseconds, 30 nanoseconds, 40 nanoseconds, 50 nanoseconds, 60 nanoseconds, 70 nanoseconds, 80 nanoseconds, 90 nanoseconds, 100 nanoseconds, 120 nanoseconds, 140 nanoseconds, 150 nanoseconds, 160 nanoseconds, 180 nanoseconds, 200 nanoseconds, 300 nanoseconds, 400 nanoseconds, 500 nanoseconds, etc. In some such implementations, the control system 206 may be capable of controlling the light source system 208 to emit a plurality of light pulses at a frequency between about 1 MHz and about 100 MHz. In other words, regardless of the wavelength(s) of light being emitted by the light source system 208, the intervals between light pulses may correspond to a frequency between about 1 MHz and about 100 MHz or more. For example, the control system 206 may be capable of controlling the light source system 208 to emit a plurality of light pulses at a frequency of about 1 MHz, about 5 MHz, about 10 MHz, about 15 MHz, about 20 MHz, about 25 MHz, about 30 MHz, about 40 MHz, about 50 MHz, about 60 MHz, about 70 MHz, about 80 MHz, about 90 MHz, about 100 MHz, etc.

In some examples, light emitted by the light source system 208 may be transmitted through an ultrasonic sensor array or through one or more substrates of a sensor stack that includes an ultrasonic sensor array. In some examples, light emitted by the light source system 208 may be transmitted through a button of a mobile device, such as the button 510 shown in FIG. 5.

In this example, block 620 involves acquiring second ultrasonic image data from the second acoustic wave emissions received by the ultrasonic sensor array. According to this implementation, block 625 involves performing an authentication process. In this example, the authentication process is based on data corresponding to both the first ultrasonic image data and the second ultrasonic image data.

For example, a control system of the mobile device 500 may be capable of comparing attribute information obtained from image data received via an ultrasonic sensor array of the apparatus 200 with stored attribute information obtained from image data that has previously been received from an authorized user. In some examples, the attribute information obtained from the received image data and the stored attribute information may include attribute information corresponding to sub-epidermal features, such as muscle tissue features, vascular features, fat lobule features or bone features.

According to some implementations, the attribute information obtained from the received image data and the stored attribute information may include information regarding fingerprint minutia. In some such implementations, the user authentication process may involve evaluating information regarding the fingerprint minutia as well as at least one other type of attribute information, such as attribute information corresponding to sub-epidermal features. According to some such examples, the user authentication process may involve evaluating information regarding the fingerprint minutia as well as attribute information corresponding to vascular features. For example, attribute information obtained from a received image of blood vessels in the finger may be compared with a stored image of blood vessels in the authorized user's finger.

The apparatus 200 that is included in the mobile device 500 may or may not include an ultrasonic transmitter, depending on the particular implementation. However, in some examples, the user authentication process may involve obtaining ultrasonic image data via insonification of the target object with ultrasonic waves from an ultrasonic transmitter. In some such examples, ultrasonic waves emitted by the ultrasonic transmitter system 210 may be transmitted through a button of a mobile device, such as the button 510 shown in FIG. 5. According to some such examples, the ultrasonic image data obtained via insonification of the target object may include fingerprint image data.

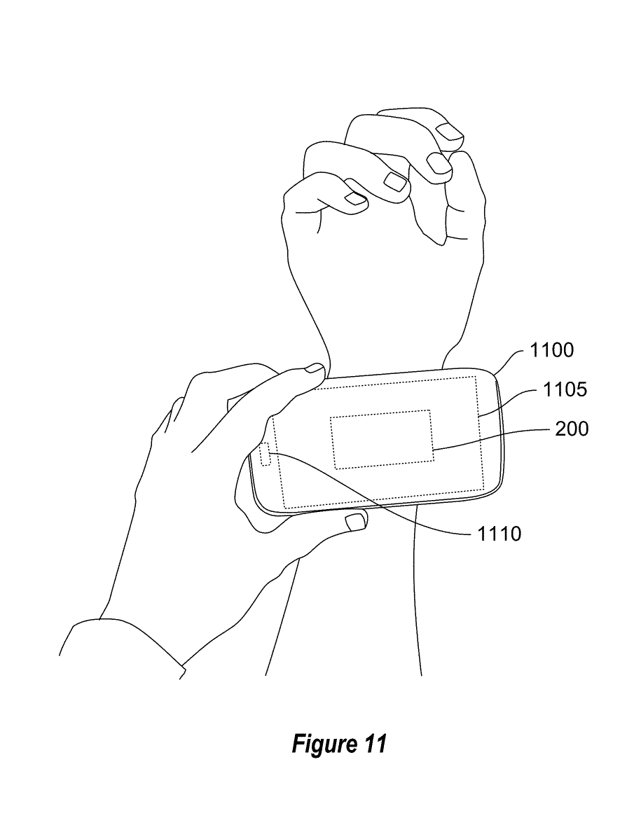

According to some implementations, the authentication process may include a liveness detection process. For example, the liveness detection process may involve detecting whether there are temporal changes of epidermal or sub-epidermal features, such as temporal changes of epidermal or sub-epidermal features caused by the flow of blood through one or more blood vessels in the target object. Some RF-acoustic imaging and/or via photoacoustic imaging implementations can detect changes in blood oxygen levels, which can provide enhanced liveness determinations. Accordingly, in some implementations, a control system may be capable of providing one or more types of monitoring, such as blood oxygen level monitoring, blood glucose level monitoring and/or heart rate monitoring. Some such implementations are described below with reference to FIG. 11 et seq.

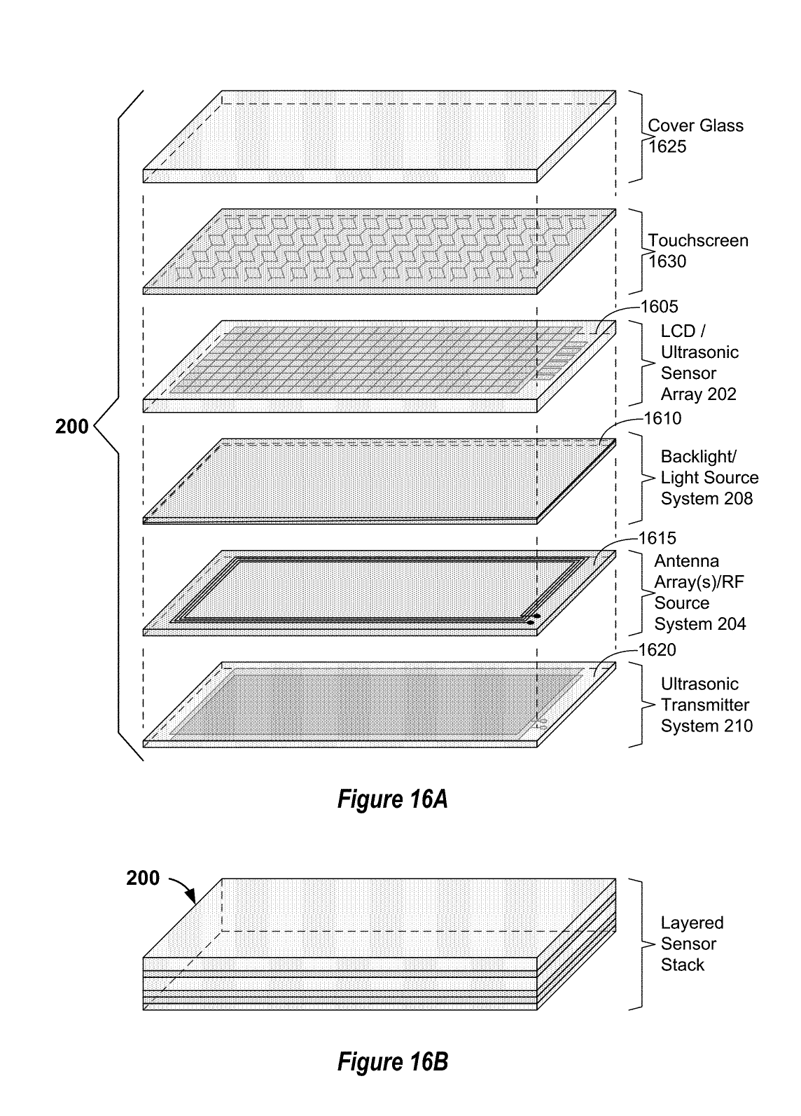

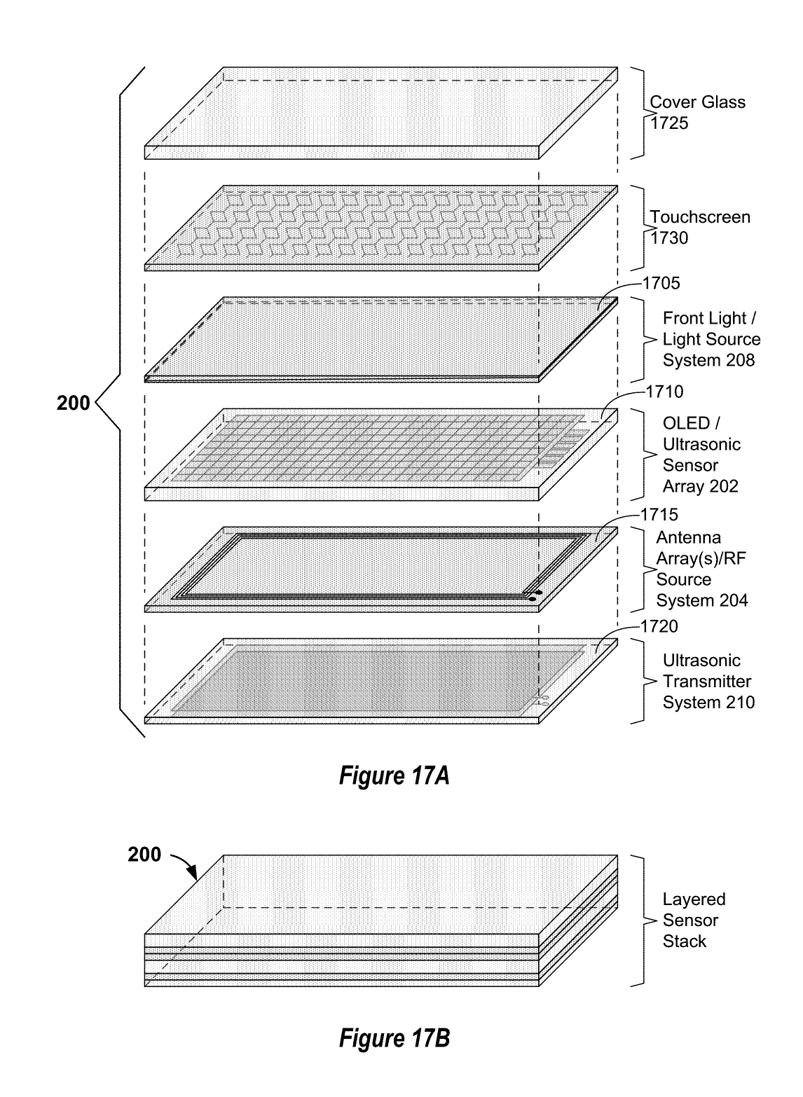

Various configurations of sensor arrays and source systems are contemplated by the inventors. In some examples, such as those described below with reference to FIGS. 16A-17B, the ultrasonic sensor array 202, the RF source system 204 and the light source system 208 may reside in different layers of the apparatus 200. However, in alternative implementations at least some sensor pixels may be integrated with display pixels.

FIG. 6B shows an example of an apparatus that includes in-cell multi-functional pixels. As with other figures disclosed herein, the numbers, types and arrangements of elements shown in FIG. 6B are only presented by way of example. In this example, the apparatus 200 includes a display 630. FIG. 6B shows an expanded view of a single pixel 635 of the display 630. In this implementation, the pixel 635 includes red, green and blue subpixels of the display 630. A control system of the apparatus 200 may be capable of controlling the red, green and blue subpixels to present images on the display 630.

According to this example, the pixel 635 also includes an optical (visible spectrum) subpixel and an infrared subpixel, both of which may be suitable for use in a light source system 208. The optical subpixel and the infrared subpixel may, for example, be laser diodes or other optical sources that are capable of emitting light suitable for inducing acoustic wave emissions inside a target object. In this example, the RF subpixel is an element of the RF source system 204, and is capable of emitting RF radiation that can induce acoustic wave emissions inside a target object.

Here, the ultrasonic subpixel is capable of emitting ultrasonic waves. In some examples, the ultrasonic subpixel may be capable of receiving ultrasonic waves and of emitting corresponding output signals. In some implementations, the ultrasonic subpixel may include one or more piezoelectric micromachined ultrasonic transducers (PMUTs), capacitive micromachined ultrasonic transducers (CMUTs), etc.

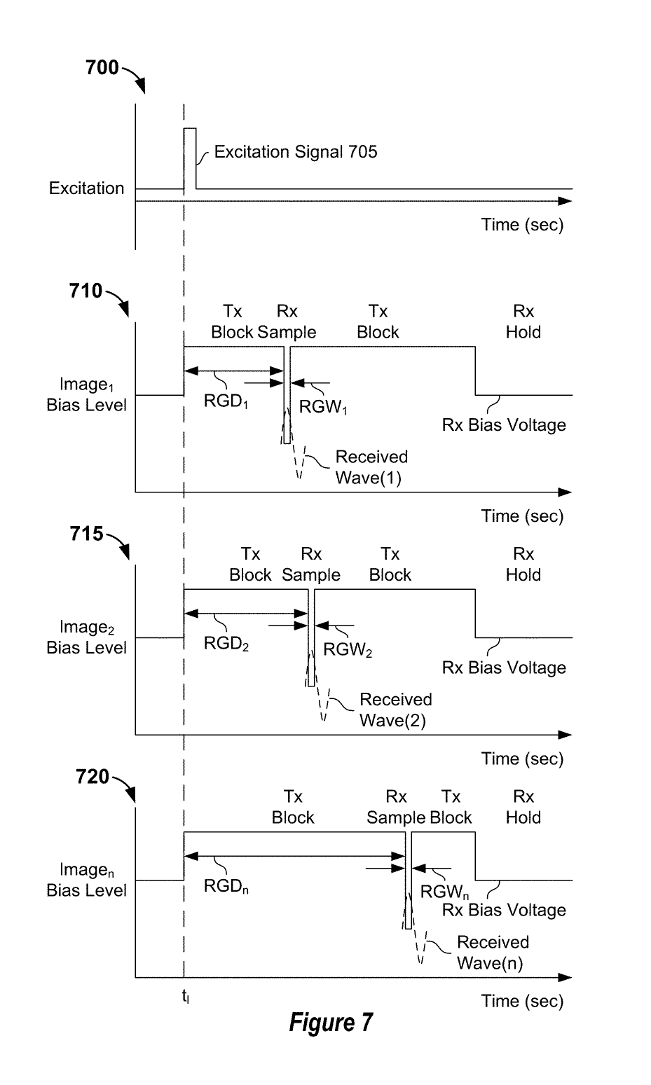

FIG. 7 shows examples of multiple acquisition time delays being selected to receive acoustic waves emitted from different depths. In these examples, each of the acquisition time delays (which are labeled range-gate delays or RGDs in FIG. 7) is measured from the beginning time t₁ of the excitation signal 705 shown in graph 700. The excitation signal 705 may, for example, correspond with RF radiation or light. The graph 710 depicts emitted acoustic waves (received wave (1) is one example) that may be received by an ultrasonic sensor array at an acquisition time delay RGD₁ and sampled during an acquisition time window (also known as a range-gate window or a range-gate width) RGW₁. Such acoustic waves will generally be emitted from a relatively shallower portion of a target object proximate, or positioned upon, a platen of the biometric system.

Graph 715 depicts emitted acoustic waves (received wave (2) is one example) that are received by the ultrasonic sensor array at an acquisition time delay RGD₂ (with RGD₂>RGD₁) and sampled during an acquisition time window RGW₂. Such acoustic waves will generally be emitted from a relatively deeper portion of the target object. Graph 720 depicts emitted acoustic waves (received wave (n) is one example) that are received at an acquisition time delay RGDₙ (with RGDₙ>RGD₂>RGD₁) and sampled during an acquisition time window of RGWₙ. Such acoustic waves will generally be emitted from a still deeper portion of the target object. Range-gate delays are typically integer multiples of a clock period. A clock frequency of 128 MHz, for example, has a clock period of 7.8125 nanoseconds, and RGDs may range from under 10 nanoseconds to over 20,000 nanoseconds. Similarly, the range-gate widths may also be integer multiples of the clock period, but are often much shorter than the RGD (e.g. less than about 50 nanoseconds) to capture returning signals while retaining good axial resolution. In some implementations, the acquisition time window (e.g. RGW) may be between less than about 10 nanoseconds to about 200 nanoseconds or more. Note that while various image bias levels (e.g. Tx block, Rx sample and Rx hold that may be applied to an Rx bias electrode) may be in the single or low double-digit volt range, the return signals may have voltages in the tens or hundreds of millivolts.

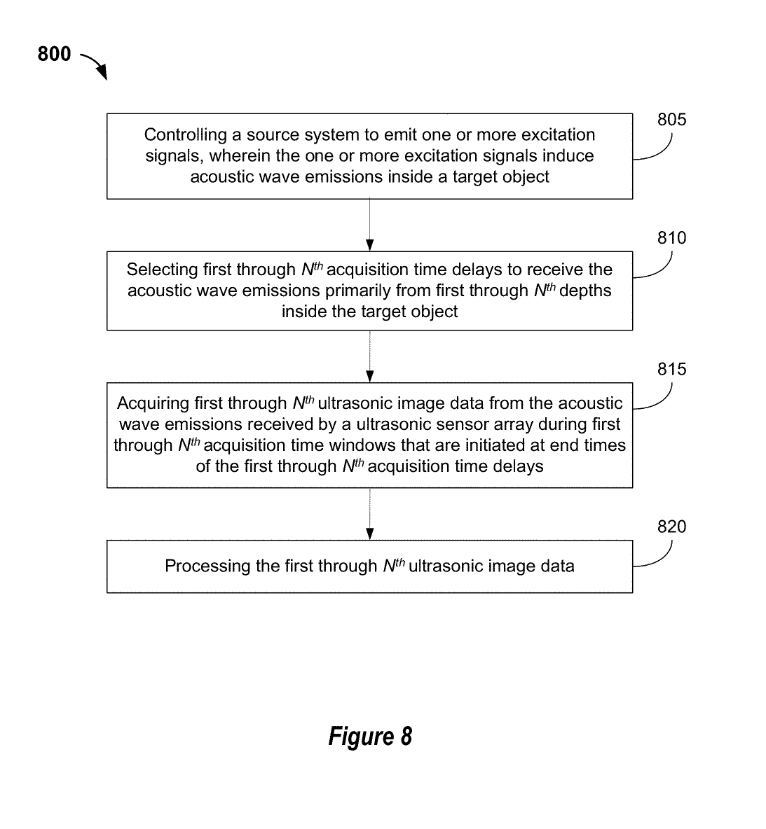

FIG. 8 is a flow diagram that provides additional examples of biometric system operations. The blocks of FIG. 8 (and those of other flow diagrams provided herein) may, for example, be performed by the apparatus 200 of FIG. 2 or by a similar apparatus. As with other methods disclosed herein, the method outlined in FIG. 8 may include more or fewer blocks than indicated. Moreover, the blocks of method 800, as well as other methods disclosed herein, are not necessarily performed in the order indicated.

Here, block 805 involves controlling a source system to emit one or more excitation signals. In this example, the one or more excitation signals induce acoustic wave emissions inside a target object in block 805. According to some examples, the control system 206 of the apparatus 200 may control the RF source system 204 to emit RF radiation in block 805. In some implementations, the control system 206 of the apparatus 200 may control the light source system 208 to emit light in block 805. According to some such implementations, the control system 206 may be capable of controlling the source system to emit at least one pulse having a duration that is in the range of about 10 nanoseconds to about 500 nanoseconds. In some such implementations, the control system 206 may be capable of controlling the source system to emit a plurality of pulses.

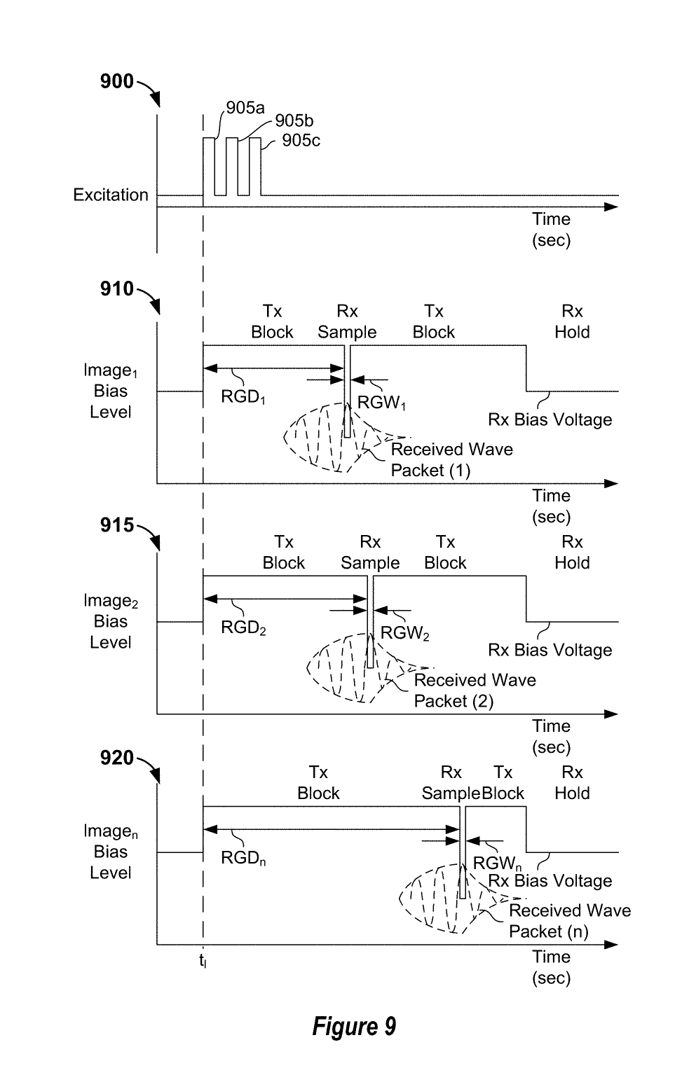

FIG. 9 shows examples of multiple acquisition time delays being selected to receive ultrasonic waves emitted from different depths, in response to a plurality of pulses. In these examples, each of the acquisition time delays (which are labeled RGDs in FIG. 9) is measured from the beginning time t₁ of the excitation signal 905a as shown in graph 900. Accordingly, the examples of FIG. 9 are similar to those of FIG. 7. However, in FIG. 9, the excitation signal 905a is only the first of multiple excitation signals. In this example, the multiple excitation signals include the excitation signals 905b and 905c, for a total of three excitation signals. In other implementations, a control system may control a source system to emit more or fewer excitation signals. In some implementations, the control system may be capable of controlling the source system to emit a plurality of pulses at a frequency between about 1 MHz and about 100 MHz.

The graph 910 illustrates ultrasonic waves (received wave packet (1) is one example) that are received by an ultrasonic sensor array at an acquisition time delay RGD₁ and sampled during an acquisition time window of RGW₁. Such ultrasonic waves will generally be emitted from a relatively shallower portion of a target object proximate to, or positioned upon, a platen of the biometric system. By comparing received wave packet (1) with received wave (1) of FIG. 7, it may be seen that the received wave packet (1) has a relatively longer time duration and a higher amplitude buildup than that of received wave (1) of FIG. 7. This longer time duration corresponds with the multiple excitation signals in the examples shown in FIG. 9, as compared to the single excitation signal in the examples shown in FIG. 7.

Graph 915 illustrates ultrasonic waves (received wave packet (2) is one example) that are received by the ultrasonic sensor array at an acquisition time delay RGD₂ (with RGD₂>RGD₁) and sampled during an acquisition time window of RGW₂. Such ultrasonic waves will generally be emitted from a relatively deeper portion of the target object. Graph 920 illustrates ultrasonic waves (received wave packet (n) is one example) that are received at an acquisition time delay RGDₙ (with RGDₙ>RGD₂>RGD₁) and sampled during an acquisition time window of RGWₙ. Such ultrasonic waves will generally be emitted from still deeper portions of the target object.

Returning to FIG. 8, in this example block 810 involves selecting first through N^{th} acquisition time delays to receive the acoustic wave emissions primarily from first through N^{th} depths inside the target object. In some such examples, the control system may be capable of selecting the first through N^{th} acquisition time delays to receive acoustic wave emissions at corresponding first through N^{th} distances from the ultrasonic sensor array. The corresponding distances may correspond to first through N^{th} depths within the target object. According to some such examples, (e.g., as shown in FIGS. 7 and 9), the acquisition time delays may be measured from a time that the light source system emits light. In some examples, the first through N^{th} acquisition time delays may be in the range of about 10 nanoseconds to over about 20,000 nanoseconds.

According to some examples, a control system (such as the control system 206) may be capable of selecting the first through N^{th} acquisition time delays. In some examples, the control system may be capable of receiving one or more of the first through N^{th} acquisition time delays (or one or more indications of depths or distances that correspond to acquisition time delays) from a user interface, from a data structure stored in memory, or by calculation of one or more depth-to-time conversions. Accordingly, in some instances the control system's selection of the first through N^{th} acquisition time delays may be according to user input, according to one or more acquisition time delays stored in memory and/or according to a calculation.

In this implementation, block 815 involves acquiring first through N^{th} ultrasonic image data from the acoustic wave emissions received by an ultrasonic sensor array during first through N^{th} acquisition time windows that are initiated at end times of the first through N^{th} acquisition time delays. According to some implementations, the first through N^{th} ultrasonic image data may be acquired during first through N^{th} acquisition time windows from a peak detector circuit disposed in each of a plurality of sensor pixels within the ultrasonic sensor array.

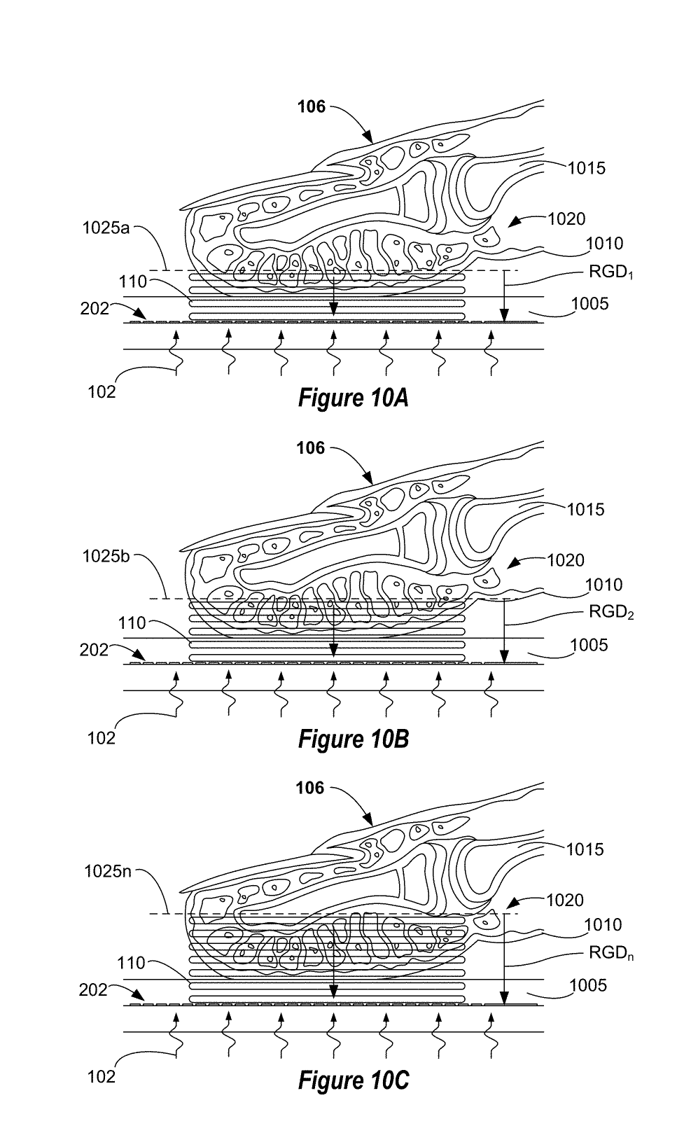

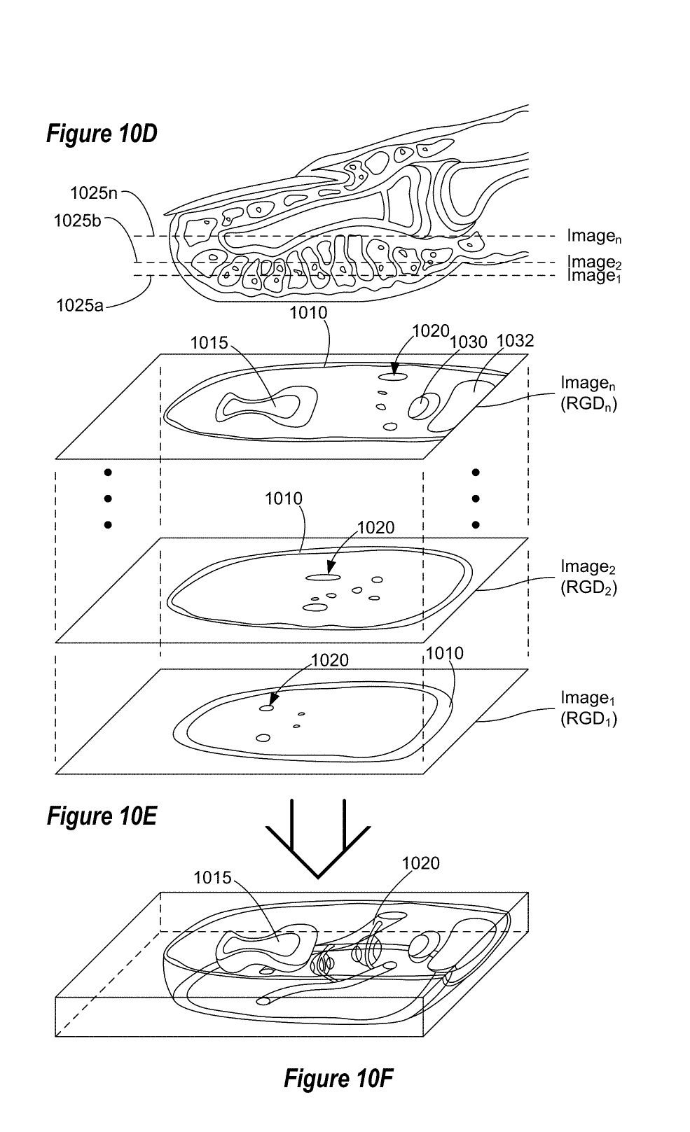

In this example, block 820 involves processing the first through N^{th} ultrasonic image data. According to some implementations block 820 may involve controlling a display to depict a two-dimensional image that corresponds with one of the first through N^{th} ultrasonic image data. In some implementations, block 820 may involve controlling a display to depict a reconstructed three-dimensional (3-D) image that corresponds with at least a subset of the first through N^{th} ultrasonic image data. Various examples are described below with reference to FIGS. 10A-10F.

FIGS. 10A-10C are examples of cross-sectional views of a target object positioned on a platen of an apparatus such as those disclosed herein. In this example, the target object is a finger 106, which is positioned on an outer surface of a platen 1005. FIGS. 10A-10C show examples of tissues and structures of the finger 106, including the epidermis 1010, bone tissue 1015, blood vasculature 1020 and various sub-epidermal tissues. In this example, incident radiation 102 has been transmitted from a light source system (not shown) through the platen 1005 and into the finger 106. Here, the incident radiation 102 has caused excitation of the epidermis 1010 and blood vasculature 1020 and resultant generation of acoustic waves 110, which can be detected by the ultrasonic sensor array 202.

FIGS. 10A-10C indicate ultrasonic image data being acquired at three different range-gate delays (RGD₁, RGD₂ and RGDₙ), which are also referred to herein as acquisition time delays, after the beginning of a time interval of excitation. The dashed horizontal lines 1025a, 1025b and 1025n in FIGS. 10A-10C indicate the depth of each corresponding image. In some examples the photo excitation may be a single pulse (e.g., as shown in FIG. 7), whereas in other examples the photo excitation may include multiple pulses (e.g., as shown in FIG. 9). FIG. 10D is a cross-sectional view of the target object illustrated in FIGS. 10A-10C. FIG. 10D show the image planes 1025a, 1025b, . . . 1025n at varying depths through which image data has been acquired.

FIG. 10E shows a series of simplified two-dimensional images that correspond with ultrasonic image data acquired by the processes shown in FIGS. 10A-10C. In this example, the simplified two-dimensional images that correspond with the image planes 1025a, 1025b and 1025n that are shown in FIG. 10D. The two-dimensional images shown in FIG. 10E provide examples of two-dimensional images corresponding with ultrasonic image data that a control system could, in some implementations, cause a display device to display.

Image₁ of FIG. 10E corresponds with the ultrasonic image data acquired using RGD₁, which corresponds with the depth 1025a shown in FIGS. 10A and 10D. Image₁ includes a portion of the epidermis 1010 and blood vasculature 1020 and also indicates structures of the sub-epidermal tissues.

Image₂ corresponds with ultrasonic image data acquired using RGD₂, which corresponds with the depth 1025b shown in FIGS. 10B and 10D. Image₂ also includes a portion of the epidermis 1010, blood vasculature 1020 and indicates some additional structures of the sub-epidermal tissues.

Imageₙ corresponds with ultrasonic image data acquired using RGDₙ, which corresponds with the depth 1025n shown in FIGS. 10C and 10D. Imageᵣ, includes a portion of the epidermis 1010, blood vasculature 1020, some additional structures of the sub-epidermal tissues and structures corresponding to bone tissue 1015. Imageₙ also includes structures 1030 and 1032, which may correspond to bone tissue 1015 and/or to connective tissue near the bone tissue 1015, such as cartilage. However, it is not clear from Image₁, Image₂ or Imageₙ what the structures of the blood vasculature 1020 and sub-epidermal tissues are or how they relate to one another.

These relationships may be more clearly seen the three-dimensional image shown in FIG. 10F. FIG. 10F shows an example of a composite image. In this example, FIG. 10F shows a composite of Image₁, Image₂ and Imageₙ, as well as additional images corresponding to depths that are between depth 1025b and depth 1025n. A three-dimensional image may be made from a set of two-dimensional images according to various methods known by those of skill in the art, such as a MATLAB® reconstruction routine or other routine that enables reconstruction or estimations of three-dimensional structures from sets of two-dimensional layer data. These routines may use spline-fitting or other curve-fitting routines and statistical techniques with interpolation to provide approximate contours and shapes represented by the two-dimensional ultrasonic image data. As compared to the two-dimensional images shown in FIG. 10E, the three-dimensional image shown in FIG. 10F more clearly represents structures corresponding to bone tissue 1015 as well as sub-epidermal structures including blood vasculature 1020, revealing vein, artery and capillary structures and other vascular structures along with bone shape, size and features.

FIG. 11 shows an example of a mobile device capable of performing some methods disclosed herein. The mobile device 1100 may be capable of various types of mobile health monitoring, such as the imaging of blood vessel patterns, the analysis of blood and/or tissue components, cancer screening, tumor imaging, imaging of other biological components and/or biomedical conditions, etc. In this example, the mobile device 1100 includes an instance of the apparatus 200 that is capable of functioning as an in-display RF-acoustic and/or photoacoustic imager. The apparatus 200 may, for example, be capable of emitting RF radiation that induces acoustic wave emissions inside a target object and of acquiring ultrasonic image data from acoustic wave emissions received by an ultrasonic sensor array. According to some examples, the apparatus 200 may be capable of emitting light that induces acoustic wave emissions inside a target object and of acquiring ultrasonic image data from acoustic wave emissions received by an ultrasonic sensor array. In some examples, the apparatus 200 may be capable of acquiring ultrasonic image data during one or more acquisition time windows that are initiated at the end time of one or more acquisition time delays.

According to some implementations, the mobile device 1100 may be capable of displaying two-dimensional and/or three-dimensional images on the display 1105 that correspond with ultrasonic image data obtained via the apparatus 200. In other implementations, the mobile device may transmit ultrasonic image data (and/or attributes obtained from ultrasonic image data) to another device for processing and/or display.

In some examples, a control system of the mobile device 1100 (which may include a control system of the apparatus 200) may be capable of selecting one or more peak frequencies of RF radiation, and/or one or more wavelengths of light, emitted by the apparatus 200. In some examples, the control system may be capable of selecting one or more peak frequencies of RF radiation and/or wavelengths of light to trigger acoustic wave emissions primarily from a particular type of material in the target object. According to some implementations, the control system may be capable of estimating a blood oxygen level and/or of estimating a blood glucose level.

In some implementations, the control system may be capable of selecting one or more peak frequencies of RF radiation and/or wavelengths of light according to user input. For example, the mobile device 1100 may allow a user or a specialized software application to enter values corresponding to one or more peak frequencies of RF radiation, or wavelengths of the light, emitted by the apparatus 200.

Alternatively or additionally, the mobile device 1100 may allow a user to select a desired function (such as estimating a blood oxygen level) and may determine one or more corresponding wavelengths of light to be emitted by the apparatus 200. For example, in some implementations, a wavelength in the mid-infrared region of the electromagnetic spectrum may be selected and a set of ultrasonic image data may be acquired in the vicinity of blood inside a blood vessel within a target object such as a finger or wrist. A second wavelength in another portion of the infrared region (e.g. near IR region) or in a visible region such as a red wavelength may be selected and a second set of ultrasonic image data may be acquired in the same vicinity as the first ultrasonic image data. A comparison of the first and second sets of ultrasonic image data, in conjunction with image data from other wavelengths or combinations of wavelengths, may allow an estimation of the blood glucose levels and/or blood oxygen levels within the target object.

In some implementations, a light source system of the mobile device 1100 may include at least one backlight or front light configured for illuminating the display 1105 and a target object. For example, the light source system may include one or more laser diodes, semiconductor lasers or light-emitting diodes. In some examples, the light source system may include at least one infrared, optical, red, green, blue, white or ultraviolet light-emitting diode or at least one infrared, optical, red, green, blue or ultraviolet laser diode. According to some implementations, the control system may be capable of controlling the light source system to emit at least one light pulse having a duration that is in the range of about 10 nanoseconds to about 500 nanoseconds. In some instances, the control system may be capable of controlling the light source system to emit a plurality of light pulses at a frequency between about 1 MHz and about 100 MHz. Alternatively or additionally, the control system may be capable of controlling an RF source system to emit RF radiation at one or more frequencies in the range of about 10 MHz to about 60 GHz or more.

In this example, the mobile device 1100 may include an ultrasonic authenticating button 1110 that includes another instance of the apparatus 200 that is capable of performing a user authentication process. In some such examples, the ultrasonic authenticating button 1110 may include an ultrasonic transmitter. According to some examples, the user authentication process may involve obtaining ultrasonic image data via insonification of a target object with ultrasonic waves from an ultrasonic transmitter and obtaining ultrasonic image data via irradiating the target object with one or more excitation signals from a source system, such as an RF source system and/or a light source system. In some such implementations, the ultrasonic image data obtained via insonification of the target object may include fingerprint image data and the ultrasonic image data obtained via irradiating the target object with one or more excitation signals may include image data corresponding to one or more sub-epidermal features, such as vascular image data.

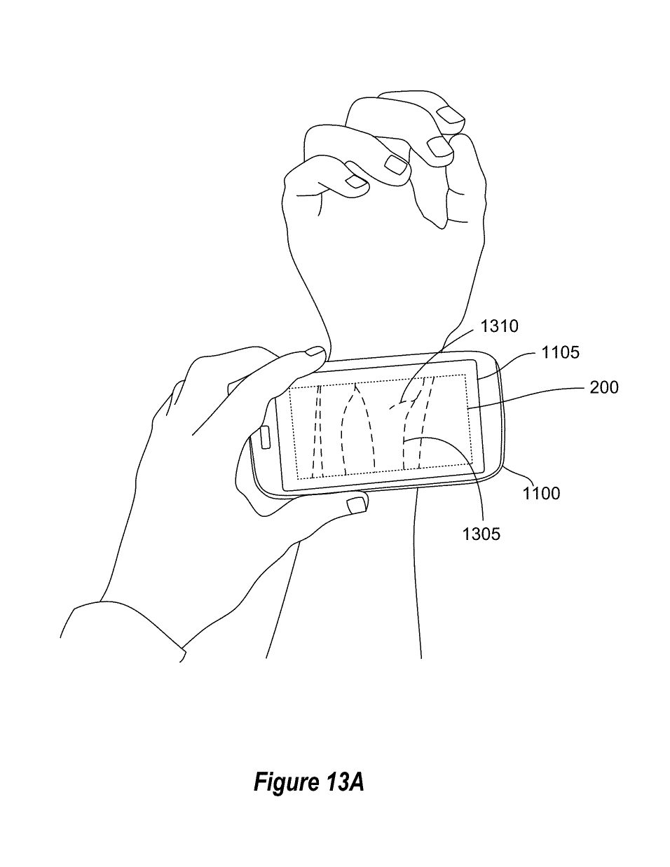

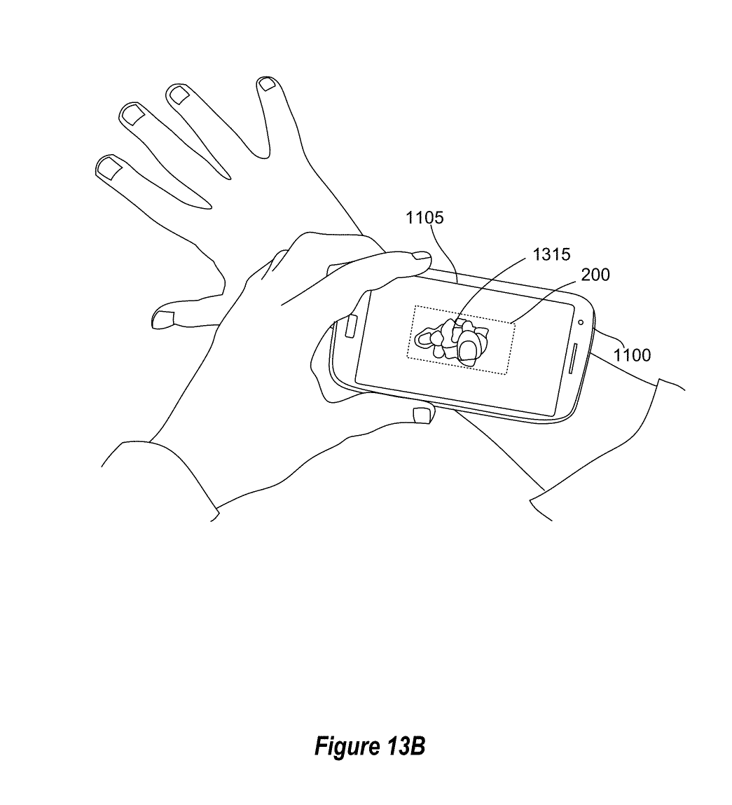

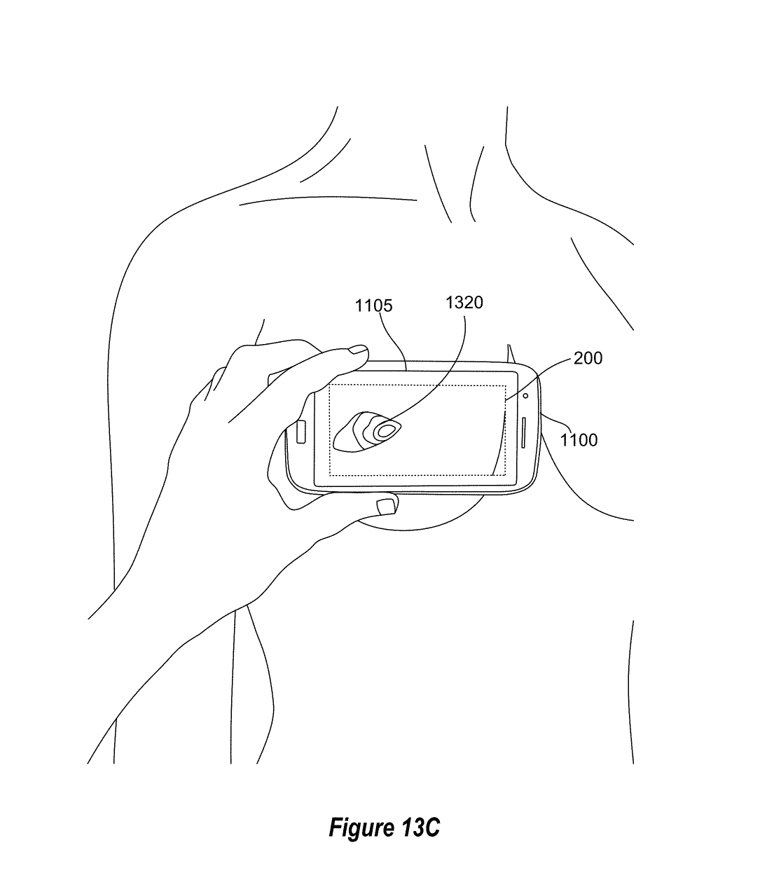

In this implementation, both the display 1105 and the apparatus 200 are on the side of the mobile device that is facing a target object, which is a wrist in this example, which may be imaged via the apparatus 200. However, in alternative implementations, the apparatus 200 may be on the opposite side of the mobile device 1100. For example, the display 1105 may be on the front of the mobile device and the apparatus 200 may be on the back of the mobile device. Some such examples are shown in FIGS. 13A-13C and are described below. According to some such implementations, the mobile device may be capable of displaying two-dimensional and/or three-dimensional images, analogous to those shown in FIGS. 10E and 10F, as the corresponding ultrasonic image data are being acquired.

In some implementations, a portion of a target object, such as a wrist or arm, may be scanned as the mobile device 1100 is moved. According to some such implementations, a control system of the mobile device 1100 may be capable of stitching together the scanned images to form a more complete and larger two-dimensional or three-dimensional image. In some examples, the control system may be capable of acquiring first and second ultrasonic image data at primarily a first depth inside a target object. The second ultrasonic image data may be acquired after the target object or the mobile device 1100 is repositioned. In some implementations, the second ultrasonic image data may be acquired after a period of time corresponding to a frame rate, such as a frame rate between about one frame per second and about thirty frames per second or more. According to some such examples, the control system may be capable of stitching together or otherwise assembling the first and second ultrasonic image data to form a composite ultrasonic image.

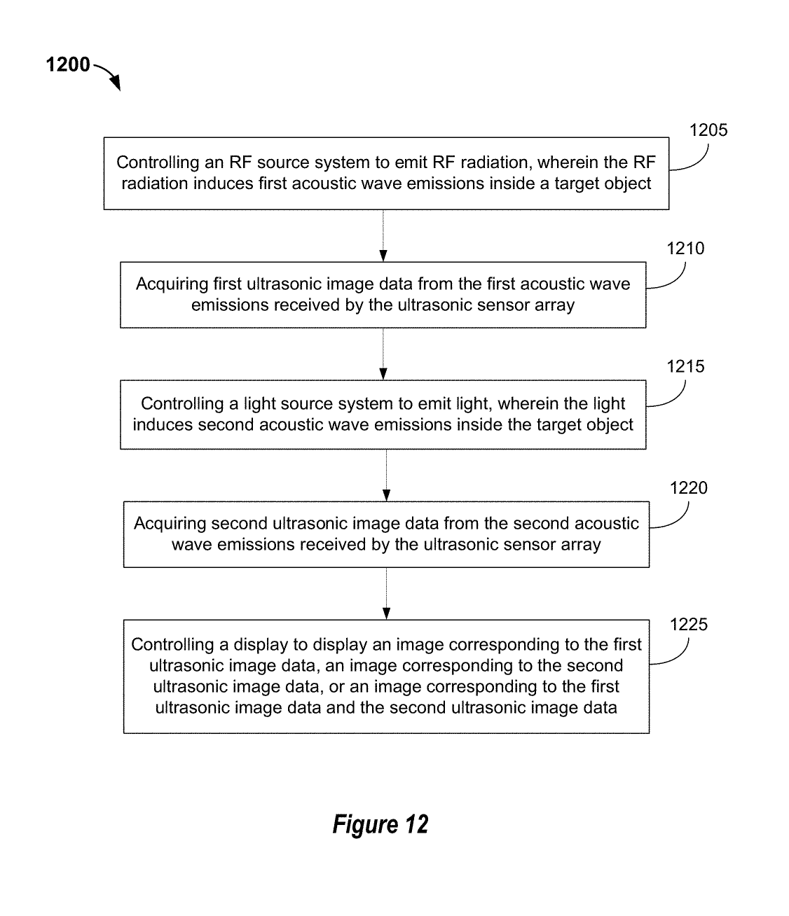

FIG. 12 is a flow diagram that provides an example of a method of obtaining and displaying ultrasonic image data via a mobile device. The mobile device may be similar to those shown in FIG. 11 or in any of FIGS. 13A-13C. As with other methods disclosed herein, the method outlined in FIG. 12 may include more or fewer blocks than indicated. Moreover, the blocks of method 1200 are not necessarily performed in the order indicated.

Here, block 1205 involves controlling an RF source system to emit RF radiation. In this example, the RF radiation induces acoustic wave emissions inside a target object in block 1205. In some implementations, the control system 206 of the apparatus 200 may control the RF source system 204 to emit RF radiation in block 1205. In some examples, the control system 206 may control the RF source system 204 to emit RF radiation at one or more frequencies in the range of about 10 MHz to about 60 GHz or more. According to some such implementations, the control system 206 may be capable of controlling the RF source system 204 to emit at least one RF radiation pulse having a duration of less than 100 nanoseconds, or less than approximately 100 nanoseconds. For example, the control system 206 may be capable of controlling the RF source system 204 to emit at least one RF radiation pulse having a duration of approximately 10 nanoseconds, 20 nanoseconds, 30 nanoseconds, 40 nanoseconds, 50 nanoseconds, 60 nanoseconds, 70 nanoseconds, 80 nanoseconds, 90 nanoseconds, 100 nanoseconds, etc.

In some examples, block 1205 (or another block of method 1200) may involve selecting a first acquisition time delay to receive the acoustic wave emissions primarily from a first depth inside the target object. In some such examples, the control system may be capable of selecting an acquisition time delay to receive acoustic wave emissions at a corresponding distance from the ultrasonic sensor array. The corresponding distance may correspond to a depth within the target object. According to some such examples, the acquisition time delay may be measured from a time that the RF source system emits RF radiation. In some examples, the acquisition time delay may be in the range of about 10 nanoseconds to about 20,000 nanoseconds.

According to some examples, a control system (such as the control system 206) may be capable of selecting the first acquisition time delay. In some examples, the control system may be capable of selecting the acquisition time delay based, at least on part, on user input. For example, the control system may be capable of receiving an indication of target depth or a distance from a platen surface of the biometric system via a user interface. The control system may be capable of determining a corresponding acquisition time delay from a data structure stored in memory, by performing a calculation, etc. Accordingly, in some instances the control system's selection of an acquisition time delay may be according to user input and/or according to one or more acquisition time delays stored in memory.

In this implementation, block 1210 involves acquiring first ultrasonic image data from the acoustic wave emissions received by an ultrasonic sensor array during a first acquisition time window that is initiated at an end time of the first acquisition time delay. Some implementations may involve controlling a display to depict a two-dimensional image that corresponds with the first ultrasonic image data. According to some implementations, the first ultrasonic image data may be acquired during the first acquisition time window from a peak detector circuit disposed in each of a plurality of sensor pixels within the ultrasonic sensor array. In some implementations, the peak detector circuitry may capture acoustic wave emissions or reflected ultrasonic wave signals during the acquisition time window. Some examples are described below with reference to FIG. 14.

In some examples, the first ultrasonic image data may include image data corresponding to one or more sub-epidermal features, such as vascular image data.

According to this implementation, block 1215 involves controlling a light source system to emit light. For example, the control system 206 may control the light source system 208 to emit light. In this example, the light induces second acoustic wave emissions inside the target object. According to some such implementations, the control system 206 may be capable of controlling the light source system 208 to emit at least one light pulse having a duration that is in the range of about 10 nanoseconds to about 500 nanoseconds or more. For example, the control system 206 may be capable of controlling the light source system 208 to emit at least one light pulse having a duration of approximately 10 nanoseconds, 20 nanoseconds, 30 nanoseconds, 40 nanoseconds, 50 nanoseconds, 60 nanoseconds, 70 nanoseconds, 80 nanoseconds, 90 nanoseconds, 100 nanoseconds, 120 nanoseconds, 140 nanoseconds, 150 nanoseconds, 160 nanoseconds, 180 nanoseconds, 200 nanoseconds, 300 nanoseconds, 400 nanoseconds, 500 nanoseconds, etc. In some such implementations, the control system 206 may be capable of controlling the light source system 208 to emit a plurality of light pulses at a frequency between about 1 MHz and about 100 MHz. In other words, regardless of the wavelength(s) of light being emitted by the light source system 208, the intervals between light pulses may correspond to a frequency between about 1 MHz and about 100 MHz or more. For example, the control system 206 may be capable of controlling the light source system 208 to emit a plurality of light pulses at a frequency of about 1 MHz, about 5 MHz, about 10 MHz, about 15 MHz, about 20 MHz, about 25 MHz, about 30 MHz, about 40 MHz, about 50 MHz, about 60 MHz, about 70 MHz, about 80 MHz, about 90 MHz, about 100 MHz, etc.

In some examples, a display may be on a first side of the mobile device and an RF source system may emit RF radiation through a second and opposing side of the mobile device. In some examples, the light source system may emit light through the second and opposing side of the mobile device.

In this example, block 1220 involves acquiring second ultrasonic image data from the second acoustic wave emissions received by the ultrasonic sensor array. According to this implementation, block 1225 involves controlling the display to display an image corresponding to the first ultrasonic image data, an image corresponding to the second ultrasonic image data, or an image corresponding to the first ultrasonic image data and the second ultrasonic image data.

In some examples, the mobile device may include an ultrasonic transmitter system. In some such examples, the ultrasonic sensor array 202 may include the ultrasonic transmitter system. In some implementations, method 1200 may involve acquiring third ultrasonic image data from insonification of the target object with ultrasonic waves emitted from the ultrasonic transmitter system. According to some such implementations, block 1225 may involve controlling the display to present an image corresponding to one or more of the first ultrasonic image data, the second ultrasonic image data and the third ultrasonic image data. In some such implementations, a control system may be capable of controlling the display to depict an image that superimposes at least two images. The at least two images may include a first image that corresponds with the first ultrasonic image data, a second image that corresponds with the second ultrasonic image data and/or a third image that corresponds with the third ultrasonic image data.

According to some implementations, the control system may be capable of selecting first through N^{th} acquisition time delays and to acquire first through N^{th} ultrasonic image data during first through N^{th} acquisition time windows after the first through N^{th} acquisition time delays. Each of the first through N^{th} acquisition time delays may, for example, correspond to first through N^{th} depths inside the target object. According to some examples, at least some of the first through N^{th} acquisition time delays may be selected to image at least one object, such as a blood vessel, a bone, fat tissue, a melanoma, a breast cancer tumor, a biological component and/or a biomedical condition.

In some examples, the control system may be capable of controlling the display to depict an image that corresponds with at least a subset of the first through N^{th} ultrasonic image data. According to some such examples, the control system may be capable of controlling a display to depict a three-dimensional (3-D) image that corresponds with at least a subset of the first through N^{th} ultrasonic image data.

FIGS. 13A-13C show examples of mobile devices imaging objects of a person's body. In the examples shown in FIGS. 13A-13C, the display 1105 is on a first side of the mobile device 1100 and at least a portion of an instance of the apparatus 200 resides on, or near, a second and opposing side of the mobile device. Accordingly, an RF source system of the apparatus 200 may emit RF radiation through the second and opposing side of the mobile device. In some implementations, a light source system also may emit light through the second and opposing side of the mobile device.

In the example shown in FIG. 13A, one or more acquisition time delays have been selected to image bones 1305 inside a patient's wrist. According to this implementation, the mobile device 1100 is capable of displaying at least a two-dimensional image on the display 1105 that corresponds with ultrasonic image data of the bones 1305 obtained via the apparatus 200. In this example, the image indicates a small fracture 1310 in one of the bones 1305.

In the example shown in FIG. 13B, multiple acquisition time delays have been selected to image a possible melanoma 1315 in a patient's skin. According to this implementation, the mobile device 1100 is capable of displaying a three-dimensional image on the display 1105 that corresponds with ultrasonic image data of the possible melanoma 1315 obtained via the apparatus 200. In some implementations, a control system of the mobile device 1100 may be capable of indicating depths and/or depth ranges of the possible melanoma 1315, e.g., via indicating different colors on the display 1105 that correspond with different depths and/or depth ranges. The depths and/or depth ranges may correspond with acquisition time delays. Knowledge of the depths and/or depth ranges of portions of the possible melanoma 1315 may aid in diagnosis, because increasing depths of a melanoma may correspond with increasingly later stages of a cancerous condition.

In the example shown in FIG. 13C, multiple acquisition time delays have been selected to image a possible tumor 1320 inside a patient's breast. According to this implementation, the mobile device 1100 is capable of displaying a three-dimensional image on the display 1105 that corresponds with ultrasonic image data of the possible tumor 1320 obtained via the apparatus 200. In some implementations, a control system of the mobile device 1100 may be capable of indicating depths and/or depth ranges of the possible tumor 1320.

FIG. 14 shows an example of a sensor pixel array. FIG. 14 representationally depicts aspects of a 4×4 pixel array 1435 of sensor pixels 1434 for an ultrasonic sensor system. Each pixel 1434 may be, for example, associated with a local region of piezoelectric sensor material (PSM), a peak detection diode (D1) and a readout transistor (M3); many or all of these elements may be formed on or in a substrate to form the pixel circuit 1436. In practice, the local region of piezoelectric sensor material of each pixel 1434 may transduce received ultrasonic energy into electrical charges. The peak detection diode D1 may register the maximum amount of charge detected by the local region of piezoelectric sensor material PSM. Each row of the pixel array 1435 may then be scanned, e.g., through a row select mechanism, a gate driver, or a shift register, and the readout transistor M3 for each column may be triggered to allow the magnitude of the peak charge for each pixel 1434 to be read by additional circuitry, e.g., a multiplexer and an A/D converter. The pixel circuit 1436 may include one or more TFTs to allow gating, addressing, and resetting of the pixel 1434.

Each pixel circuit 1436 may provide information about a small portion of the object detected by the ultrasonic sensor system. While, for convenience of illustration, the example shown in FIG. 14 is of a relatively coarse resolution, ultrasonic sensors having a resolution on the order of 500 pixels per inch or higher may be configured with an appropriately scaled structure. The detection area of the ultrasonic sensor system may be selected depending on the intended object of detection. For example, the detection area may range from about 5 mm×5 mm for a single finger to about 3 inches×3 inches for four fingers. Smaller and larger areas, including square, rectangular and non-rectangular geometries, may be used as appropriate for the target object.

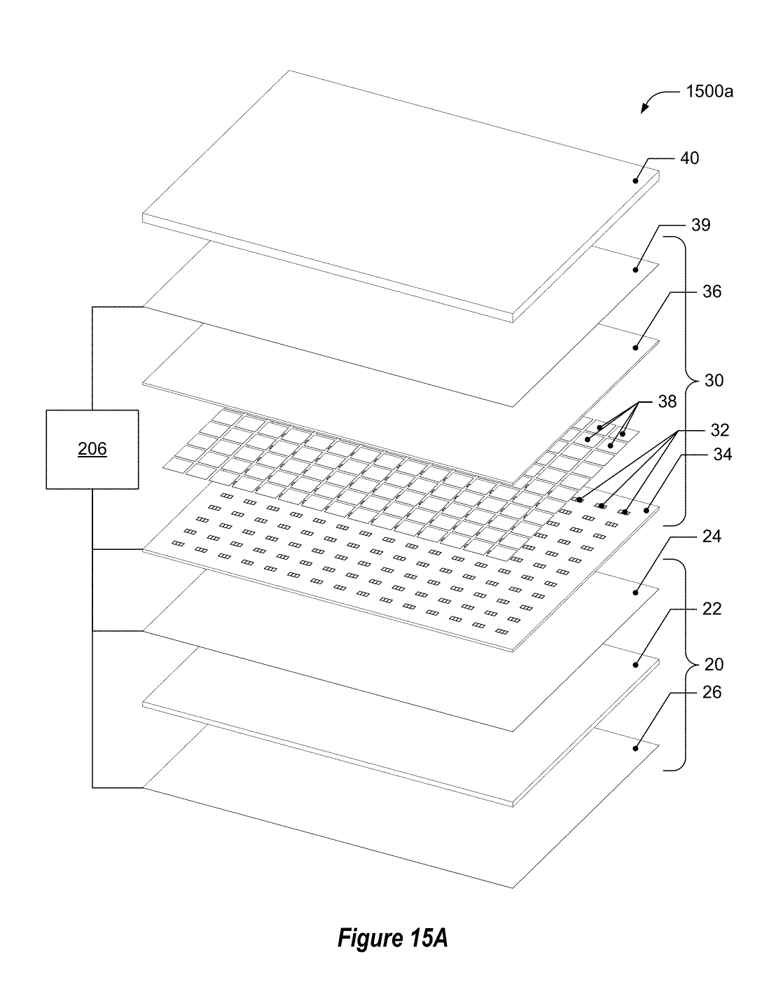

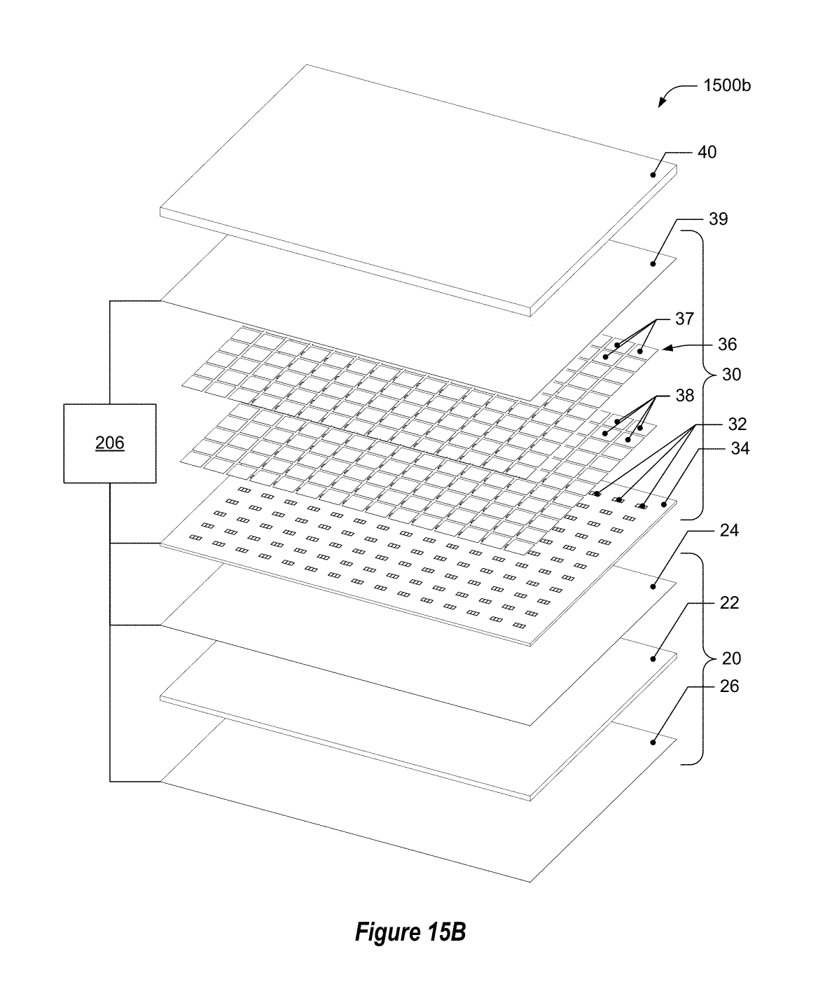

FIG. 15A shows an example of an exploded view of an ultrasonic sensor system. In this example, the ultrasonic sensor system 1500a includes an ultrasonic transmitter 20 and an ultrasonic receiver 30 under a platen 40. According to some implementations, the ultrasonic receiver 30 may be an example of the ultrasonic sensor array 202 that is shown in FIG. 2 and described above. In some implementations, the ultrasonic transmitter 20 may be an example of the optional ultrasonic transmitter system 210 that is shown in FIG. 2 and described above. The ultrasonic transmitter 20 may include a substantially planar piezoelectric transmitter layer 22 and may be capable of functioning as a plane wave generator. Ultrasonic waves may be generated by applying a voltage to the piezoelectric layer to expand or contract the layer, depending upon the signal applied, thereby generating a plane wave. In this example, the control system 206 may be capable of causing a voltage that may be applied to the planar piezoelectric transmitter layer 22 via a first transmitter electrode 24 and a second transmitter electrode 26. In this fashion, an ultrasonic wave may be made by changing the thickness of the layer via a piezoelectric effect. This ultrasonic wave may travel towards a finger (or other object to be detected), passing through the platen 40. A portion of the wave not absorbed or transmitted by the object to be detected may be reflected so as to pass back through the platen 40 and be received by the ultrasonic receiver 30. The first and second transmitter electrodes 24 and 26 may be metallized electrodes, for example, metal layers that coat opposing sides of the piezoelectric transmitter layer 22.

The ultrasonic receiver 30 may include an array of sensor pixel circuits 32 disposed on a substrate 34, which also may be referred to as a backplane, and a piezoelectric receiver layer 36. In some implementations, each sensor pixel circuit 32 may include one or more TFT elements, electrical interconnect traces and, in some implementations, one or more additional circuit elements such as diodes, capacitors, and the like. Each sensor pixel circuit 32 may be configured to convert an electric charge generated in the piezoelectric receiver layer 36 proximate to the pixel circuit into an electrical signal. Each sensor pixel circuit 32 may include a pixel input electrode 38 that electrically couples the piezoelectric receiver layer 36 to the sensor pixel circuit 32.

In the illustrated implementation, a receiver bias electrode 39 is disposed on a side of the piezoelectric receiver layer 36 proximal to platen 40. The receiver bias electrode 39 may be a metallized electrode and may be grounded or biased to control which signals may be passed to the array of sensor pixel circuits 32. Ultrasonic energy that is reflected from the exposed (top) surface of the platen 40 may be converted into localized electrical charges by the piezoelectric receiver layer 36. These localized charges may be collected by the pixel input electrodes 38 and passed on to the underlying sensor pixel circuits 32. The charges may be amplified or buffered by the sensor pixel circuits 32 and provided to the control system 206.

The control system 206 may be electrically connected (directly or indirectly) with the first transmitter electrode 24 and the second transmitter electrode 26, as well as with the receiver bias electrode 39 and the sensor pixel circuits 32 on the substrate 34. In some implementations, the control system 206 may operate substantially as described above. For example, the control system 206 may be capable of processing the amplified signals received from the sensor pixel circuits 32.

The control system 206 may be capable of controlling the ultrasonic transmitter 20 and/or the ultrasonic receiver 30 to obtain ultrasonic image data, e.g., by obtaining fingerprint images. Whether or not the ultrasonic sensor system 1500a includes an ultrasonic transmitter 20, the control system 206 may be capable of obtaining attribute information from the ultrasonic image data. In some examples, the control system 206 may be capable of controlling access to one or more devices based, at least in part, on the attribute information. The ultrasonic sensor system 1500a (or an associated device) may include a memory system that includes one or more memory devices. In some implementations, the control system 206 may include at least a portion of the memory system. The control system 206 may be capable of obtaining attribute information from ultrasonic image data and storing the attribute information in the memory system. In some implementations, the control system 206 may be capable of capturing a fingerprint image, obtaining attribute information from the fingerprint image and storing attribute information obtained from the fingerprint image (which may be referred to herein as fingerprint image information) in the memory system. According to some examples, the control system 206 may be capable of capturing a fingerprint image, obtaining attribute information from the fingerprint image and storing attribute information obtained from the fingerprint image even while maintaining the ultrasonic transmitter 20 in an “off” state.

In some implementations, the control system 206 may be capable of operating the ultrasonic sensor system 1500a in an ultrasonic imaging mode or a force-sensing mode. In some implementations, the control system 206 may be capable of maintaining the ultrasonic transmitter 20 in an “off” state when operating the ultrasonic sensor system in a force-sensing mode. The ultrasonic receiver 30 may be capable of functioning as a force sensor when the ultrasonic sensor system 1500a is operating in the force-sensing mode. In some implementations, the control system 206 may be capable of controlling other devices, such as a display system, a communication system, etc. In some implementations, the control system 206 may be capable of operating the ultrasonic sensor system 1500a in a capacitive imaging mode.

The platen 40 may be any appropriate material that can be acoustically coupled to the receiver, with examples including plastic, ceramic, sapphire, metal and glass. In some implementations, the platen 40 may be a cover plate, e.g., a cover glass or a lens glass for a display. Particularly when the ultrasonic transmitter 20 is in use, fingerprint detection and imaging can be performed through relatively thick platens if desired, e.g., 3 mm and above. However, for implementations in which the ultrasonic receiver 30 is capable of imaging fingerprints in a force detection mode or a capacitance detection mode, a thinner and relatively more compliant platen 40 may be desirable. According to some such implementations, the platen 40 may include one or more polymers, such as one or more types of parylene, and may be substantially thinner. In some such implementations, the platen 40 may be tens of microns thick or even less than 10 microns thick.

Examples of piezoelectric materials that may be used to form the piezoelectric receiver layer 36 include piezoelectric polymers having appropriate acoustic properties, for example, an acoustic impedance between about 2.5 MRayls and 5 MRayls. Specific examples of piezoelectric materials that may be employed include ferroelectric polymers such as polyvinylidene fluoride (PVDF) and polyvinylidene fluoride-trifluoroethylene (PVDF-TrFE) copolymers. Examples of PVDF copolymers include 60:40 (molar percent) PVDF-TrFE, 70:30 PVDF-TrFE, 80:20 PVDF-TrFE, and 90:10 PVDR-TrFE. Other examples of piezoelectric materials that may be employed include polyvinylidene chloride (PVDC) homopolymers and copolymers, polytetrafluoroethylene (PTFE) homopolymers and copolymers, and diisopropylammonium bromide (DIPAB).

The thickness of each of the piezoelectric transmitter layer 22 and the piezoelectric receiver layer 36 may be selected so as to be suitable for generating and receiving ultrasonic waves. In one example, a PVDF planar piezoelectric transmitter layer 22 is approximately 28 μm thick and a PVDF-TrFE receiver layer 36 is approximately 12 μm thick. Example frequencies of the ultrasonic waves may be in the range of 5 MHz to 30 MHz, with wavelengths on the order of a millimeter or less.

FIG. 15B shows an exploded view of an alternative example of an ultrasonic sensor system. In this example, the piezoelectric receiver layer 36 has been formed into discrete elements 37. In the implementation shown in FIG. 15B, each of the discrete elements 37 corresponds with a single pixel input electrode 38 and a single sensor pixel circuit 32. However, in alternative implementations of the ultrasonic sensor system 1500b, there is not necessarily a one-to-one correspondence between each of the discrete elements 37, a single pixel input electrode 38 and a single sensor pixel circuit 32. For example, in some implementations there may be multiple pixel input electrodes 38 and sensor pixel circuits 32 for a single discrete element 37.

FIGS. 15A and 15B show example arrangements of ultrasonic transmitters and receivers in an ultrasonic sensor system, with other arrangements possible. For example, in some implementations, the ultrasonic transmitter 20 may be above the ultrasonic receiver 30 and therefore closer to the object(s) 25 to be detected. In some implementations, the ultrasonic transmitter may be included with the ultrasonic sensor array (e.g., a single-layer transmitter and receiver). In some implementations, the ultrasonic sensor system may include an acoustic delay layer. For example, an acoustic delay layer may be incorporated into the ultrasonic sensor system between the ultrasonic transmitter 20 and the ultrasonic receiver 30. An acoustic delay layer may be employed to adjust the ultrasonic puke timing, and at the same time electrically insulate the ultrasonic receiver 30 from the ultrasonic transmitter 20. The acoustic delay layer may have a substantially uniform thickness, with the material used for the delay layer and/or the thickness of the delay layer selected to provide a desired delay in the time for reflected ultrasonic energy to reach the ultrasonic receiver 30. In doing so, the range of time during which an energy pulse that carries information about the object by virtue of having been reflected by the object may be made to arrive at the ultrasonic receiver 30 during a time range when it is unlikely that energy reflected from other parts of the ultrasonic sensor system is arriving at the ultrasonic receiver 30. In some implementations, the substrate 34 and/or the platen 40 may serve as an acoustic delay layer.

FIG. 16A shows examples of layers of an apparatus according to one example. In this implementation, the stack of the apparatus 200 includes a substrate 1605 on which a display and an ultrasonic sensor array 202 reside. The display is a liquid crystal display (LCD) in this example. Here, a backlight residing on the substrate 1610 includes a light source system 208. In this example, an RF source system 204, which includes one or more RF antenna arrays, resides on the substrate 1615. In this implementation, an ultrasonic transmitter system 210 resides on the substrate 1620. This implementation includes a cover glass 1625 and a touchscreen 1630. FIG. 16B shows an example of a layered sensor stack that includes the layers shown in FIG. 16A.

FIG. 17A shows examples of layers of an apparatus according to another example. Here, the apparatus 200 includes a front light and a light source system 208 residing on the substrate 1705. In this implementation, a display and an ultrasonic sensor array 202 reside on a substrate 1710. The display is an organic light-emitting diode (OLED) display in this example. In this example, an RF source system 204, which includes one or more RF antenna arrays, resides on the substrate 1715. In this implementation, an ultrasonic transmitter system 210 resides on the substrate 1720. This implementation includes a cover glass 1725 and a touchscreen 1730. FIG. 17B shows an example of a layered sensor stack that includes the layers shown in FIG. 17A.

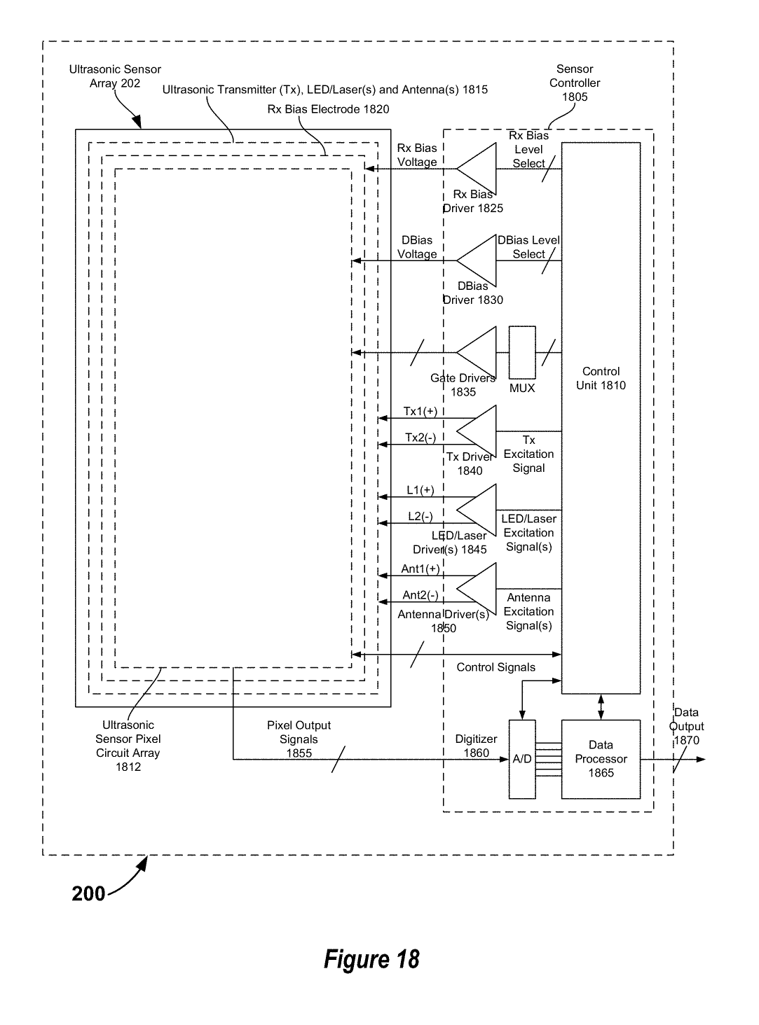

FIG. 18 shows example elements of an apparatus such as those disclosed herein. In this example, the sensor controller 1805 is configured for controlling the apparatus 200. Accordingly, the sensor controller 1805 includes at least a portion of the control system 206 that is shown in FIG. 2 and described elsewhere herein. In this example, the layer 1815 includes an ultrasonic transmitter, LEDs and/or laser diodes, and antennas. In this implementation, the ultrasonic transmitter is an instance of an ultrasonic transmitter system 210, the LEDs and laser diodes are elements of a light source system 208, and the antennas are elements of an RF source system 204. According to this implementation, the ultrasonic sensor array 202 includes the ultrasonic sensor pixel circuit array 1812. In this example, the sensor controller 1805 is configured for controlling the ultrasonic sensor array 202, the ultrasonic transmitter, the LEDs and laser diodes, and the antennas.

In the example shown in FIG. 18, the sensor controller 1805 includes a control unit 1810, a receiver bias driver 1825, a DBias voltage driver 1830, gate drivers 1835, transmitter driver 1840, LED/laser driver 1845, one or more antenna drivers 1850, one or more digitizers 1860 and a data processor 1865. Here, the receiver bias driver 1825 is configured to apply a bias voltage to the receiver bias electrode 1820 according to a receiver bias level control signal from the control unit 1810. In this example, the DBias voltage driver 1830 is configured to apply a diode bias voltage to the ultrasonic sensor pixel circuit array 1812 according to a DBias level control signal from the control unit 1810.

In this implementation, the gate drivers 1835 control the range gate delay and range gate windows of the ultrasonic sensor array 202 according to multiplexed control signals from the control unit 1810. According to this example, the transmitter driver 1840 controls the ultrasonic transmitter according to ultrasonic transmitter excitation signals from the control unit 1810. In this example, the LED/laser driver 1845 controls the LEDs and laser diodes to emit light according to LED/laser excitation signals from the control unit 1810. Similarly, in this example, one or more antenna drivers 1850 may control the antennas to emit RF radiation according to antenna excitation signals from the control unit 1810.

According to this implementation, the ultrasonic sensor array 202 may be configured to send analog pixel output signals 1855 to the digitizer 1860. The digitizer 1860 converts the analog signals to digital signals and provides the digital signals to the data processor 1865. The data processor 1865 may process the digital signals according to control signals from the control unit 1810 and outputs processed signals 1870. In some implementations, the data processor 1865 may filter the digital signals, subtract a background image, amplify a pixel value, adjust a grayscale level, and/or shift an offset value. In some implementations, the data processor 1865 may perform an image processing function and/or perform a higher level function such as execute a matching routine or perform an authentication process to authenticate a user.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module that may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

1. An apparatus, comprising:
an ultrasonic sensor array;
a radio frequency (RF) source system; and
a control system capable of:
controlling the RF source system to emit RF radiation, wherein the RF radiation induces first acoustic wave emissions inside a target object; and
acquiring first ultrasonic image data from the first acoustic wave emissions received by the ultrasonic sensor array from the target object.

2. The apparatus of claim 1, wherein the control system is further capable of selecting a first acquisition time delay for the reception of acoustic wave emissions primarily from a first depth inside the target object.

3. A mobile device that includes the apparatus of claim 1.

4. The apparatus of claim 1, wherein the RF source system includes an antenna array capable of emitting RF radiation at one or more frequencies in the range of about 10 MHz to about 60 GHz.

5. The apparatus of claim 1, wherein RF radiation emitted from the RF source system is emitted as one or more pulses, each pulse having a duration less than about 100 nanoseconds.

6. The apparatus of claim 1, wherein the RF source system includes a broad-area antenna array capable of irradiating the target object with either substantially uniform RF radiation or with focused RF radiation at a target depth.

7. The apparatus of claim 1, wherein the RF source system includes one or more loop antennas, one or more dipole antennas, one or more microstrip antennas, one or more slot antennas, one or more patch antennas, one or more lossy waveguide antennas, or one or more millimeter wave antennas, the antennas residing on one or more substrates that are coupled to the ultrasonic sensor array.

8. The apparatus of claim 1, further comprising a light source system, wherein the control system is capable of:
controlling the light source system to emit light that induces second acoustic wave emissions inside the target object; and
acquiring second ultrasonic image data from the acoustic wave emissions received by the ultrasonic sensor array from the target object.

9. The apparatus of claim 8, wherein the light source system is capable of emitting one or more of infrared (IR) light, visible light (VIS) or ultraviolet (UV) light.

10. The apparatus of claim 8, further comprising a substrate, wherein the ultrasonic sensor array resides in or on the substrate and at least a portion of the light source system is coupled to the substrate.

11. The apparatus of claim 10, wherein IR light, VIS light or UV light from the light source system is transmitted through the substrate.

12. The apparatus of claim 10, wherein RF radiation emitted by the RF source system is transmitted through the substrate.

13. The apparatus of claim 10, further comprising a display, wherein subpixels of the display are coupled to the substrate.

14. The apparatus of claim 13, wherein the control system is further capable of controlling the display to depict a two-dimensional image that corresponds with the first ultrasonic image data or the second ultrasonic image data.

15. The apparatus of claim 13, wherein the control system is further capable of controlling the display to depict an image that superimposes a first image that corresponds with the first ultrasonic image data and a second image that corresponds with the second ultrasonic image data.

16. The apparatus of claim 8, wherein light emitted from the light source system is emitted as one or more pulses, each pulse having a duration less than about 100 nanoseconds.

17. The apparatus of claim 1, further comprising a display, wherein subpixels of the display are adapted to detect one or more of infrared light, visible light, UV light, ultrasonic waves, or acoustic wave emissions.

18. The apparatus of claim 1, wherein RF radiation emitted by the RF source system is transmitted through the ultrasonic sensor array.

19. The apparatus of claim 1, wherein the control system is further capable of selecting first through N^{th} acquisition time delays and of acquiring first through N^{th} ultrasonic image data during first through N^{th} acquisition time windows after the first through N^{th} acquisition time delays, each of the first through N^{th} acquisition time delays corresponding to first through N^{th} depths inside the target object.

20. The apparatus of claim 19, further comprising a display, wherein the control system is further capable of controlling the display to depict a three-dimensional image that corresponds with at least a subset of the first through N^{th} ultrasonic image data.

21. The apparatus of claim 1, wherein the first ultrasonic image data is acquired during a first acquisition time window from a peak detector circuit disposed in each of a plurality of sensor pixels within the ultrasonic sensor array.

22. The apparatus of claim 1, wherein the ultrasonic sensor array and a portion of the RF source system are configured in one of an ultrasonic button, a display module, or a mobile device enclosure.

23. The apparatus of claim 1, further comprising an ultrasonic transmitter system, wherein the control system is further capable of acquiring second ultrasonic image data from insonification of the target object with ultrasonic waves emitted from the ultrasonic transmitter system.

24. The apparatus of claim 23, wherein ultrasonic waves emitted from the ultrasonic transmitter system are emitted as one or more pulses, each pulse having a duration less than about 100 nanoseconds.

25. The apparatus of claim 1, further comprising a light source system and an ultrasonic transmitter system, wherein the control system is further capable of controlling the light source system and the ultrasonic transmitter system, and wherein the control system is further capable of acquiring second acoustic wave emissions, via the ultrasonic sensor array, from the target object in response to RF radiation emitted from the RF source system, light emitted from the light source system, or ultrasonic waves emitted by the ultrasonic transmitter system.

26. A mobile device, comprising:
an ultrasonic sensor array;
a display;
a radio frequency (RF) source system;
a light source system; and
a control system capable of:
controlling the RF source system to emit RF radiation, wherein the RF radiation induces first acoustic wave emissions inside a target object;
acquiring first ultrasonic image data from the first acoustic wave emissions received by the ultrasonic sensor array from the target object;
controlling the light source system to emit light that induces second acoustic wave emissions inside the target object;
acquiring second ultrasonic image data from the acoustic wave emissions received by the ultrasonic sensor array from the target object; and
controlling the display to display an image corresponding to the first ultrasonic image data, an image corresponding to the second ultrasonic image data, or an image corresponding to the first ultrasonic image data and the second ultrasonic image data.

27. The mobile device of claim 26, wherein:
the display is on a first side of the mobile device; and
the RF source system emits RF radiation through a second and opposing side of the mobile device.

28. The mobile device of claim 26, wherein the light source system emits light through the second and opposing side of the mobile device.

29. The mobile device of claim 26, further comprising an ultrasonic transmitter system, wherein the control system is further capable of:
acquiring third ultrasonic image data from insonification of the target object with ultrasonic waves emitted from the ultrasonic transmitter system; and
controlling the display to display an image corresponding to one or more of the first ultrasonic image data, the second ultrasonic image data or the third ultrasonic image data.

30. The mobile device of claim 29, wherein the control system is further capable of controlling the display to depict an image that superimposes at least two images selected from a group comprising: a first image that corresponds with the first ultrasonic image data; a second image that corresponds with the second ultrasonic image data; and a third image that corresponds with the third ultrasonic image data.

31. The mobile device of claim 29, wherein the ultrasonic sensor array includes the ultrasonic transmitter system.

32. The mobile device of claim 26, wherein the control system is further capable of:
selecting first through N^{th} acquisition time delays and to acquire first through N^{th} ultrasonic image data during first through N^{th} acquisition time windows after the first through N^{th} acquisition time delays, each of the first through N^{th} acquisition time delays corresponding to first through N^{th} depths inside the target object; and
controlling the display to depict an image that corresponds with at least a subset of the first through N^{th} ultrasonic image data.

33. The mobile device of claim 32, wherein the first through N^{th} acquisition time delays are selected to image at least one object selected from a list of objects consisting of a blood vessel, a bone, fat tissue, a melanoma, a breast cancer tumor, a biological component, and a biomedical condition.

34. An apparatus, comprising:
an ultrasonic sensor array;
a radio frequency (RF) source system;
a light source system; and
control means for:
controlling the RF source system to emit RF radiation, wherein the RF radiation induces first acoustic wave emissions inside a target object;
acquiring first ultrasonic image data from the first acoustic wave emissions received by the ultrasonic sensor array from the target object;
controlling the light source system to emit light, wherein the light induces second acoustic wave emissions inside the target object;
acquiring second ultrasonic image data from the second acoustic wave emissions received by the ultrasonic sensor array from the target object; and
performing an authentication process based on data corresponding to both the first ultrasonic image data and the second ultrasonic image data.

35. The apparatus of claim 34, wherein the ultrasonic sensor array, the RF source system and the light source system reside, at least in part, in a button area of a mobile device.

36. The apparatus of claim 34, wherein the authentication process comprises a liveness detection process.

37. The apparatus of claim 34, wherein the control means includes means for performing one or more types of monitoring selected from a list of monitoring types consisting of blood oxygen level monitoring, blood glucose level monitoring, and heartrate monitoring.

38. A method of acquiring ultrasonic image data, the method comprising:
controlling a radio frequency (RF) source system to emit RF radiation, wherein the RF radiation induces first acoustic wave emissions inside a target object; and
acquiring, via an ultrasonic sensor array, first ultrasonic image data from the first acoustic wave emissions received by the ultrasonic sensor array from the target object.

39. The method of claim 38, further comprising:
controlling a light source system to emit light that induces second acoustic wave emissions inside the target object; and
acquiring, via the ultrasonic sensor array, second ultrasonic image data from the acoustic wave emissions received by the ultrasonic sensor array from the target object.

40. The method of claim 39, further comprising controlling a display to display an image corresponding to the first ultrasonic image data, an image corresponding to the second ultrasonic image data, or an image corresponding to the first ultrasonic image data and the second ultrasonic image data.

41. The method of claim 39, further comprising performing an authentication process based on data corresponding to both the first ultrasonic image data and the second ultrasonic image data.

42. A non-transitory medium having software stored thereon, the software including instructions for controlling one or more devices to perform a method of acquiring ultrasonic image data, the method comprising:
controlling a radio frequency (RF) source system to emit RF radiation, wherein the RF radiation induces first acoustic wave emissions inside a target object; and
acquiring, via an ultrasonic sensor array, first ultrasonic image data from the first acoustic wave emissions received by the ultrasonic sensor array from the target object.

43. The non-transitory medium of claim 42, wherein the method further comprises:
controlling a light source system to emit light that induces second acoustic wave emissions inside the target object; and
acquiring, via the ultrasonic sensor array, second ultrasonic image data from the acoustic wave emissions received by the ultrasonic sensor array from the target object.

44. The non-transitory medium of claim 43, wherein the method further comprises controlling a display to display an image corresponding to the first ultrasonic image data, an image corresponding to the second ultrasonic image data, or an image corresponding to the first ultrasonic image data and the second ultrasonic image data.

45. The non-transitory medium of claim 43, wherein the method further comprises performing an authentication process based on data corresponding to both the first ultrasonic image data and the second ultrasonic image data.

46. The apparatus of claim 1, further comprising a platen coupled to the ultrasonic sensor array, wherein the target object is positioned on a surface of the platen.
